(19) United States
(12) Patent Application Publication
(10) Pub. No.: US 2002/0048785 A1
(43) Pub. Date: Apr. 25, 2002

(54) Novel polypeptides within the tumor necrosis factor receptor superfamily and uses therefor

(75) Inventor: Douglas A. Holtzman, Jamaica Plain, MA (US)

Correspondence Address: ANITA L. MEIKLEJOHN, PH.D., Fish & Richardson P.C., 225 Franklin Street, Boston, MA, 02110-2804, US

(73) Assignee: Millennium Pharmaceuticals, Inc. a Delaware corporation

(21) Appl. No.: 09/757,421

(22) Filed: Jan. 10, 2001

Related U.S. Application Data
(63) Continuation of application No. 08/843,652, filed on Apr. 16, 1997, now abandoned.

(51) Int. Cl. C07K 14/705; C07H 21/04; C12P 21/02; C12N 5/06

(52) U.S. Cl. 435/69.1; 435/325; 435/320.1; 530/350; 536/23.5

(57) ABSTRACT

The present invention relates to the discovery and characterization of two novel polypeptides within the tumor necrosis factor (TNF) receptor superfamily. The first receptor of the invention, Tango-63d, encodes a 440 amino acid polypeptide and the second receptor of the invention, Tango-63e, encodes a 411 amino acid polypeptide.

The invention encompasses nucleic acid molecules encoding Tango-63d and Tango-63e or mutant forms thereof which encode dysfunctional receptor polypeptides, vectors containing these nucleic acid molecules, cells harboring recombinant DNA molecules encoding Tango-63d and/or Tango-63e or mutant forms thereof, host fusion proteins which include functional or dysfunctional Tango-63d or Tango-63e, transgenic animals which express Tango-63d and/or Tango-63e, screening methods and therapeutic methods employing the nucleic acid molecules and polypeptides described above, substantially purified Tango-63d, substantially purified Tango-63e and therapeutic compositions containing these nucleic acid molecules and polypeptides.

### BACKGROUND OF THE INVENTION

In multicellular organisms, homeostasis is maintained by balancing the rate of cell proliferation against the rate of cell death. This balance is important in pathophysiologic contexts (for example, in the elimination of virally-infected and radiation-damaged cells). Cell proliferation is influenced by numerous growth factors and the expression of proto-oncogenes, which typically encourage progression through the cell cycle. In contrast, numerous events, including the expression of tumor suppressor genes, can lead to an arrest of cellular proliferation.

In differentiated cells, a particular form of cell death called apoptosis (or programmed cell death (PCD)) is carried out when an internal suicide program is activated. This program can be initiated by a variety of external signals as well as signals that are generated within the cell in response to, for example, genetic damage. Thus, apoptosis of a cell or a group of cells is presumably beneficial to the organism as a whole. For many years, the magnitude of apoptotic cell death was not appreciated because the dying cells are quickly eliminated by phagocytes, without an inflammatory response.

The mechanisms that mediate apoptosis have been intensively studied. These mechanisms involve the activation of endogenous proteases, loss of mitochondrial function, and structural changes such as disruption of the cytoskeleton, cell shrinkage, membrane blebbing, and nuclear condensation, which occurs as the cell's DNA is degraded. Initially, large fragments of DNA (of about 50 kb) are produced, and subsequent cleavage between the nucleosomes produces smaller fragments that appear as a “ladder”following electrophoresis through an agarose gel.

The various signals that trigger apoptosis are thought to bring about these events by converging on a common cell death pathway that is regulated by the expression of genes that are highly conserved from worms, such as C. elegans, to humans. In fact, invertebrate model systems have been invaluable tools in identifying and characterizing the genes that control apoptosis. Through the study of invertebrates and more evolved animals, numerous genes that are associated with cell death have been identified, but the way in which their products interact to execute the apoptotic program is poorly understood.

Currently, four cell surface receptors are known to initiate an apoptotic signal: tumor necrosis factor receptor 1 (TNFR-1, also known as p55-R); the Fas receptor (which is also called CD95 or APO-1) (Boldin et al., Cell 85:803, 1996; Muzio et al., Cell 85:817, 1996); Death Receptor 3 (DR-3 (Chinnaiyan et al., Science 274:990-992, 1996)), which is also known as WSL-1 (Kitson et al., Nature 384:372-375, 1996) or APO-3 (Marsters et al., Current Biol. 6:1669-1676, 1996); and Death Receptor 4 (DR-4; Pan et al., Science 276:111-113, 1997), which binds the APO2/TRAIL ligand.

The Fas/APO-1 receptor and TNFR-1 are classified as members of the TNF/nerve growth factor receptor family and both share an intracellular region of homology designated the “death domain” (Boldin et al., supra; Muzio et al., supra). The TNF/nerve growth factor receptor family is extremely large, and contains molecules that differ in their binding specificities; not all of the molecules in this family bind TNF. Furthermore, the regions that are homologous from one family member to another vary. Two family members may have homologous sequence in the ectodomain, but not in the death domain, or vice-versa.

The death domain of the Fas/APO-1 receptor interacts with FADD (Fas-associating protein with death domain, also known as MORT1) and RIP (receptor interacting protein), forming a complex that, when joined by Caspase-8, constitutes the Fas/APO-1 death-inducing signalling complex (Boldin et al., supra; Muzio et al., supra). The interaction between Fas/APO-1 and FADD is mediated by their respective C-terminal death domains (Chinnaiyan et al., Cell 81:505-512, 1995).

A second complex that is thought to be involved in cell death forms in association with the intracellular portion of TNFR-1, and includes Caspase-8, TRADD (TNFR-1-associated death domain protein), and FADD/MORT1 (Boldin et al.,supra; Muzio et al., supra).

Just as not all members of the TNF receptor family bind TNF (see above), not all members contain a death domain. For example, a receptor termed TNFR-2 is a 75 kDa receptor for the TNF ligand that is not believed to contain a death domain. Thus, this receptor may activate an alternative intracellular signalling pathway that may or may not lead to apoptosis (WO 96/34095; Smith et al., Cell 76:959-962, 1994).

The factors that are known to bind TNFR-1 include TNF-α and TNF-β (also known as lymphotoxin-α), which are related members of a broad family of polypeptide mediators, collectively known as cytokines, that includes the interferons, interleukins, and growth factors (Beutler and Cerami, Ann. Rev. Immunol., 7:625-655, 1989). A subset of these polypeptides are classified as TNF-related cytokines and, in addition to TNF-α and TNF-β include LT-β and ligands for the Fas and 4-1BB receptors.

TNF-α and TNF-β were first recognized for their anti-tumor activities, but are now known as pleiotropic cytokines that play a role in many biological processes. For example, TNF-α is believed to mediate immunostimulation, autoimmune disease, graft rejection, anti-viral responses, septic shock, cerebral malaria, cytotoxicity, protective responses to ionizing radiation, and growth regulation. TNF-β which is produced by activated lymphocytes, exhibits similar but not identical biological activities. TNF-β elicits tumor necrosis, mediates anti-viral responses, activates polymorphonuclear leukocytes, and induces the expression of MHC class I antigens and adhesion molecules on endothelial cells.

### SUMMARY OF THE INVENTION

The present invention relates to the discovery and characterization of two novel polypeptides with similarity to members of the TNF receptor superfamily. The first, Tango-63d, is a 440 amino acid polypeptide, and the second, Tango-63e, is a 411 amino acid polypeptide that is identical to Tango-63d, with the exception of a deletion of amino acids 183-211.

The invention encompasses nucleic acid molecules encoding Tango-63d and Tango-63e, vectors containing these nucleic acid molecules, cells harboring recombined DNA encoding Tango-63d and/or Tango-63e, fusion proteins that include Tango-63d and/or Tango-63e, transgenic animals that express Tango-63d and/or Tango-63e, and recombinant knock-out animals that fail to express Tango-63d and/or Tango-63e.

By “isolated nucleic acid molecule” is meant a nucleic acid molecule that is separated from either the 5′ or the 3′ coding sequence with which it is immediately contiguous in the naturally occurring genome of an organism. An isolated nucleic acid molecule is also referred to as “recombinant nucleic acid molecule.”

The nucleic acid molecules of the invention can be inserted into transcription and/or translation vectors, as described below, which will facilitate expression of the insert. The nucleic acid molecules and the polypeptides they encode can be used directly as diagnostic or therapeutic agents, or (in the case of a polypeptide) can be used to generate antibodies that, in turn, are therapeutically and/or diagnostically useful. Accordingly, expression vectors containing a nucleic acid of the invention, cells transfected with these vectors, the polypeptides expressed, and antibodies generated, against either the entire polypeptide or an antigenic fragment thereof, are among the preferred embodiments.

As used herein, the term “transfected cell” means any cell into which (or into an ancestor of which) has been introduced, by means of recombinant DNA techniques, a nucleic acid encoding a polypeptide of the invention (e.g., a Tango-63d polypeptide or a Tango-63e polypeptide).

As used herein, both “protein” and “polypeptide”mean any chain of amino acid residues, regardless of length or post-translational modification (e.g., glycosylation or phosphorylation). The polypeptides of the invention are referred to as “substantially pure,” meaning that they are at least 60% by weight (dry weight) the polypeptide of interest, e.g., a Tango-63d polypeptide or a Tango-63e polypeptide. Preferably, the polypeptide is at least 75%, more preferably at least 90%, and most preferably at least 99%, by weight, the polypeptide of interest. Purity can be measured by any appropriate standard method, e.g., column chromatography, polyacrylamide gel electrophoresis, or HPLC analysis. The polypeptide can be a naturally occurring, synthetic, or a recombinant molecule consisting of a hybrid with one portion, for example, being encoded by all or part of a Tango-63 gene, and a second portion being encoded by all or part of a second gene. For example, the polypeptide can be fused to a hexa-histidine tag to facilitate purification of bacterially expressed protein, or to a hemagglutinin (HA) tag to facilitate purification of protein expressed in eukaryotic cells. The HA tag corresponds to an epitope derived from the influenza hemagglutinin protein (Wilson et al., Cell 37:767, 1984). The polypeptides of the invention can also be fused to another compound (such as polyethylene glycol) that will increase the half life of the polypeptide within the circulation. Similarly, the receptor polypeptide can be fused to a heterologous polypeptide such as the Fc region of an IgG molecule, or a leader or secretory sequence.

The polypeptides of the invention can be chemically synthesized, produced by recombinant techniques from a prokaryotic or eukaryotic host (for example, by bacterial, yeast, higher plant, insect, and mammalian cells in culture), or they can be purified from tissues in which they are naturally expressed, according to standard biochemical methods of purification.

The polypeptides of the present invention can be employed to identifying putative ligands to which the polypeptides bind. These ligands can be identified, for example, by transfecting a cell population with an appropriate vector from which the polypeptide is expressed, and exposing that cell to various putative ligands. The ligands tested could include those that are known to interact with members of the TNF receptor superfamily, as well as additional small molecules, cell supernatants, extracts, or other natural products. The polypeptide can also be used to screen an expression library according to standard techniques. This is not to say that the polypeptides of the invention must interact with another molecule in order to exhibit biological activity; the polypeptides may function in a ligand-independent manner.

In the event a ligand is identified, one could then determine whether that ligand acts as a full or partial agonist or antagonist of the polypeptide of the invention using no more than routine pharmacological assays.

Also included in the invention are “functional polypeptides,” which possess one or more of the biological functions or activities of Tango-63d or Tango-63e. These functions or activities are described in detail below and concern, primarily, induction of apoptosis by, for example, binding some or all of the proteins which normally bind to Tango-63d or Tango-63e. A functional polypeptide is also considered within the scope of the invention if it serves as an immunogen for production of antibodies that specifically bind to Tango-63d or Tango-63e. In many cases, functional polypeptides retain one or more domains present in the naturally-occurring form of the polypeptide. For example, a functional polypeptide can posses one or more of the Tango-63 domains, for example, an extracellular domain, a transmembrane domain, and an intracellular domain. It is well within the abilities of skilled artisans to determine whether a polypeptide, regardless of size, retains the functional activity of a polypeptide of the invention.

The functional polypeptides can contain a primary amino acid sequence that has been modified from those disclosed herein. Preferably these modifications consist of conservative amino acid substitutions, as described herein. When the polypeptides of the invention are administered to a patient, they may be given in a membrane-bound or a soluble, circulating form. Typically, the soluble form of the polypeptide will lack the transmembrane domain. Soluble polypeptides may include any number of leader sequences at the 5′ end; the purpose of these leader sequences being, primarily, to allow expression in a eukaryotic system (see, for example, U.S. Pat. No. 5,082,783).

The members of a pair of molecules (for example, an antibody-antigen pair or a receptor-ligand pair) are said to “specifically bind” to each other if they bind to each other with greater affinity than to other molecules, even those that are structurally or functionally related to a member of the specific binding pair.

The invention also encompasses compounds which modulate the expression or activity of Tango-63d and/or Tango-63e, including small molecules (i.e., molecules with a molecular weight below about 500), large molecules (i.e., molecules with a molecular weight above about 500), and nucleic acid molecules that can be used to inhibit the expression of these genes (for example, antisense and ribozyme molecules) or to enhance their expression (for example, expression constructs that place nucleic acid sequences encoding either Tango-63d or Tango-63e under the control of a strong promoter system), and transgenic animals that express a Tango-63 transgene.

Tango-63d and/or Tango-63e function can be altered either by altering the expression of Tango-63d and/or Tango-63e (i.e., altering the amount of Tango-63d and/or Tango-63e present in a given cell) or by altering the activity of Tango-63d and/or Tango-63e.

The invention includes methods for treating disorders characterized by aberrant expression or activity of Tango-63d and/or Tango-63e. These methods entail administering a molecule which increases or decreases; as appropriate, expression of Tango-63d and/or Tango-63e.

The invention encompasses methods of treatment including a method of treating a patient who has a disorder associated with an abnormal rate of apoptotic cell death by administering a compound that modulates the expression of Tango-63d and/or Tango-63e (at the DNA, mRNA or protein level, e.g., by altering mRNA splicing) or the activity of Tango-63d and/or Tango-63e. Examples of such compounds include small molecules, antisense nucleic acid molecules, ribozymes, and molecules that specifically interact with the polypeptide and thereby act as full or partial agonists or antagonists of its activity.

Disorders that can be treated by altering the expression or activity of the polypeptides of the invention include disorders associated with either an abnormally high or an abnormally low rate or apoptotic cell death (as described further hereinbelow). In addition, T cell mediated diseases, including acquired immune deficiency syndrome (AIDS), autoimmune diseases such as systemic lupus erythrematosis, rheumatoid arthritis, and type I diabetes, septic shock, cerebral malaria, graft rejection, cytotoxicity, cachexia, and inflammation are considered amenable to treatment by altering the expression or activity of a polypeptide of the invention.

A patient who has a disorder associated with an abnormally high rate of apoptotic cell death can be treated by the administration of: a ligand (for example, a naturally occurring or synthetic ligand) that antagonizes Tango-63d or Tango-63e; a compound that decreases the expression of Tango-63d or Tango-63e; a compound that decreases the activity of Tango-63d or Tango-63e; an expression vector that contains a nucleic acid molecule that encodes a nonfunctional Tango-63; or a nonfunctional Tango-63 polypeptide itself. Preferably, the nonfunctional polypeptide will bind any naturally occurring ligand(s) of Tango-63d or Tango-63e or otherwise interfere with the ability of the polypeptides to transduce a signal. Accordingly, the invention features therapeutic compositions that contain the compounds or ligands described above.

Conversely, a patient who has a disorder associated with an abnormally low rate of apoptotic cell death can be treated by the administration of: a ligand (for example, a naturally occurring or synthetic ligand) that activates Tango-63d or Tango-63e (i.e., a ligand that acts as a full or partial agonist of Tango-63d or Tango-63e); a compound that increases the expression of Tango-63d or Tango-63e; a compound that increases the activity of Tango-63d or Tango-63e; an expression vector that contains a nucleic acid molecule encoding Tango-63d or Tango-63e, or by administering either or both of the polypeptides directly to the patient's cells (either in viva or ex viva). These methods are described more fully below.

Certain disorders are associated with an increased number of surviving cells, which are produced and continue to survive or proliferate when apoptosis is inhibited. These disorders include cancer, particularly follicular lymphomas, carcinomas associated with mutations in p53, and hormone-dependent tumors such as breast cancer, prostate cancer, and ovarian cancer. As described in the example below, Tango-63 has been mapped to a position that is located in the most frequently lost region of chromosome 8, between markers D8S133 and NEFL. As described in the example below, this region has been implicated in the etiology of numerous cancers, including prostate cancer, colon cancer, non-small cell lung cancer, breast cancer, head and neck cancer, hepatocarcinoma, and bladder cancer.

Additional disorders that are associated with an increased number of surviving cells include autoimmune disorders (such as systemic lupus erythematosus and immune-mediated glomerulonephritis), and viral infections (such as those caused by herpesviruses, poxviruses, and adenoviruses).

Populations of cells are often depleted in the event of viral infection, with perhaps the most dramatic example being the cell depletion caused by the human immunodeficiency virus (HIV). Surprisingly, most T cells that die during HIV infections do not appear to be infected with HIV. Although a number of explanations have been proposed, recent evidence suggests that stimulation of the CD4 receptor results in the enhanced susceptibility of uninfected T cells to undergo apoptosis.

A wide variety of neurological diseases are characterized by the gradual loss of specific sets of neurons. Such disorders are referred to as neurodegenerative diseases and include Alzheimer's disease, Parkinson's disease, amyotrophic lateral sclerosis (ALS), Huntington's disease, retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses.

Two common disorders associated with cell death are myocardial infarction (which is commonly referred to as a “heart attack”) and cerebral ischemia (which is commonly referred to as “stroke”). In both of these disorders, cells within the central area of ischemia, which is produced in the event of acute loss of blood flow, appear to die rapidly as a result of necrosis. However, outside the central ischemic zone, cells die over a more protracted time period and, morphologically, appear to die by apoptosis.

The present invention encompasses methods and compositions for the diagnostic evaluation, typing, and prognosis of disorders associated with apoptotic cell death and disorders related to abnormal expression or activity or Tango-63d or Tango-63e. The disorder can be associated with either an increase or a decrease in the incidence of apoptotic cell death. For example, the nucleic acid molecules of the invention can be used as diagnostic hybridization probes to detect, for example, expression of Tango-63d or Tango-63e. Such methods can be used to classify cells by their level of Tango-63d or Tango-63e expression. For example, higher Tango-63d or Tango-63e expression may be associated with a higher rate of apoptosis. The present invention further provides for diagnostic kits for the practice of such methods.

In particular, the invention described below encompasses Tango-63d or Tango-63e polypeptides corresponding to functional domains of Tango-63d or Tango-63e (e.g., the death domain), mutated, truncated, or deleted polypeptides that retain at least one of the functional activities of Tango-63d or Tango-63e (for example, a polypeptide in which one or more amino acid residues have been substituted, deleted from, or added to the death domain without destroying the ability of the mutant Tango-63d or Tango-63e polypeptides to induce apoptosis, and fusion proteins (as described below).

Polypeptides that exhibit at least 70%, preferably at least 80%, more preferably at least 90%, and most preferably at least 95% of the activity of the Tango-63d or Tango-63e polypeptides described herein are considered within the scope of the invention.

The invention encompasses nucleic acids and polypeptides that have a sequence that is substantially identical to a Tango-63d or Tango-63e nucleic acid or polypeptide. The term “substantially identical” refers to a polypeptide or nucleic acid having a sequence that is at least 85%, preferably at least 90%, more preferably at least 95%, and most preferably at least 98% or 99% or more identical to the sequence of a reference amino acid or nucleic acid sequence. For polypeptides, the length of the reference polypeptide sequence will generally be at least 16 amino acids, at least 20 amino acids, at least 25 amino acids, or preferably at least 35 amino acids. For nucleic acids, the length of the reference nucleic acid sequence will generally be at least 50 nucleotides, at least 60 nucleotides, at least 75 nucleotides, or at least 90 nucleotides.

Sequence identity can be measured using sequence analysis software (e.g., Sequence Analysis Software Package of the Genetics Computer Group, University of Wisconsin Biotechnology Center, 1710 University Avenue, Madison, Wis. 53705) with the default parameters specified therein.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference polypeptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide which is 50% identical to the reference polypeptide over its entire length. Of course, many other polypeptides will meet the same criteria.

The reference nucleic acid or polypeptide can be a naturally-occurring molecule, for example, a Tango-63d-encoding nucleic acid molecule, a Tango-63e-encoding nucleic acid molecule, a Tango-63d polypeptide, or a Tango-63e polypeptide.

A transgenic animal is any animal containing cells that bear genetic information received, directly or indirectly, by deliberate genetic manipulation at the subcellular level, such as DNA received by microinjection or by infection with recombinant virus. Thus, animals of the invention are those with one or more cells that contain a recombinant DNA molecule of the invention and, in this context, the term “animal” denotes all animals except Homo sapiens. Farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats) are especially preferred.

It is also preferred that the nucleic acid molecule becomes integrated with the animal's chromosomes, but the use of DNA sequences that replicate extrachromosomally, such as might be engineered into yeast artificial chromosomes (YACs) or human artificial chromosomes (HACs), are also contemplated.

Transgenic animals include animals in which the genetic information has been taken up and integrated into a germ line cell. These animals typically have the ability to transfer the genetic information to their offspring. If the offspring in fact possess some or all of the genetic information delivered to the parent animal, then they, too, are transgenic animals.

In another embodiment, the invention features methods of identifying compounds that modulate apoptotic cell death by modulating the expression or activity of Tango-63d and/or Tango-63e by assessing the expression or activity of Tango-63d and/or Tango-63e in the presence and absence of the compound. A difference in the level of expression or activity of Tango-63d or Tango-63e in the presence of the compound (compared with the level of expression or activity in the absence of the compound) indicates that the compound is capable of modulating the expression or activity of Tango-63d or Tango-63e and thereby useful in, for example, modulating apoptotic cell death. Expression can be assessed either at the level of gene expression (e.g., by measuring mRNA) or protein expression by techniques that are well known to skilled artisans. The activity of Tango-63d or Tango-63e can be assessed functionally, i.e., by assaying the ability of the compound to inhibit apoptosis following activation of the Tango-63d or Tango-63e receptor complexes.

The invention features an isolated nucleic acid molecule comprising a nucleotide sequence encoding a polypeptide that is at least 85% identical to SEQ ID NO:2; and an isolated nucleic acid molecule comprising a nucleotide sequence encoding a polypeptide that is at least 85% identical to SEQ ID NO:4.

In other aspect, the invention features: an isolated nucleic acid molecule that includes the nucleotide sequence of SEQ ID NO:1, and that encodes the amino acid sequence of SEQ ID NO:2; an isolated nucleic acid molecule that includes the nucleotide sequence of SEQ ID NO:3, and that encodes the amino acid sequence of SEQ ID NO:4; an isolated nucleic acid molecule that includes the molecule deposited with the American Type Culture Collection and assigned accession number 98368; and an isolated nucleic acid molecule that includes the molecule deposited with the American Type Culture Collection and assigned accession number 98367.

In another aspect, the invention features a vector that includes an above-described nucleic acid molecule. In various specific embodiments, the vector is an expression vector, and can include a regulatory element such as the cytomegalovirus hCMV immediate early gene, the early promoter of SV40 adenovirus, the late promoter of SV40 adenovirus, the lac system, the trp system, the TAC system, the TRC system, the major operator and promoter regions of phage X, the control regions of fd coat protein, the promoter for 3-phosphoglycerate kinase, the promoters of acid phosphatase, and the promoters of the yeast α-mating factors. The vector can also include a regulatory element that directs tissue-specific expression, a reporter gene such as a gene encoding β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). The vector can be a plasmid or a virus, such as a retrovirus.

In another aspect, the invention features a genetically engineered host cell, particularly a eukaryotic cell, which includes a vector, as described above.

In another aspect, the invention features a chimeric polypeptide that contains a polypeptide encoded by one or more of the nucleic acid molecules described above and a heterologous polypeptide (i.e. a polypeptide that has a sequence other than those described above as polypeptides of the invention).

In other aspects, the invention features an antibody that specifically binds Tango-63d and an antibody that specifically binds Tango-63e.

In yet another aspect, the invention features a transgenic animal harboring a nucleic acid molecule described above.

The invention also features a method for determining whether a patient has a disorder associated with an abnormal rate of apoptotic cell death. The method is carried out by quantitating the level of expression of Tango-63d or Tango-63e in a biological sample (e.g., a tumor sample) obtained from the patient. Expression can be assessed by examining the level of mRNA encoding Tango-63d or Tango-63e or the level of Tango-63d or Tango-63e protein. Methods of quantitating mRNA and protein are well known in the art of molecular biology. Methods useful in the present invention include RNAse protection assays, Northern blot analyses, the polymerase chain reaction (PCR, particularly, RT-PCR), and, to assess the level of protein expression, Western blot analyses.

The invention also features a method for determining whether a patient has a disorder associated with a mutation in a gene encoding Tango-63d or Tango-63e. The method is carried out by examining the nucleic acid sequence of Tango-63d or Tango-63e in a sample of DNA obtained from a patient.

The invention also features a method of treating a patient who has a disorder associated with abnormal activity of the Tango-63d or Tango-63e complex. The method is carried out by administering to the patient a compound that modulates the expression or activity of Tango-63d or Tango-63e. The compound can be, for example, a compound that acts as a full or partial agonist of Tango-63d or Tango-63e (which would be administered to increase the activity of Tango-63d or Tango-63e) or as a full or partial antagonist of Tango-63d or Tango-63e (which would be administered to decrease the activity of Tango-63d or Tango-63e). The compound could be a small molecule. To decrease the expression of Tango-63d or Tango-63e, an antisense nucleic acid molecule, or a ribozyme can be administered.

The invention also features therapeutic compositions which include the compounds that are used in the methods of treatment described above. The compounds identified as useful can be naturally occurring or synthetic.

In another aspect, the invention features a method for treating a patient who has a disorder associated with abnormal activity of the Tango-63d or Tango-63e by administering to the patient a compound that mediates oligomerization between Tango-63d or Tango-63e and other molecules that may assemble to form an active complex. These molecules can include TRADD, MORT1, and Caspase-8, or homologues thereof.

The patient who is treated can have any disorder associated with an abnormal level of apoptotic cell death, including acquired immune deficiency syndrome (AIDS), a neurodegenerative disorder, a myelodysplastic syndrome, an ischemic injury, a toxin-induced injury, or a cancer.

The invention also features a method of treating a patient who has a disorder associated with excessive apoptotic cell death by administering to the patient Tango-63d and/or Tango-63e nucleic acid molecules or the Tango-63d and/or Tango-63e polypeptides.

In another aspect, the invention features a method of identifying a compound that modulates expression of Tango-63d and/or Tango-63e by assessing the expression of Tango-63d or Tango-63e in the presence and absence of the compound.

The invention also features a method of treating a patient who has an abnormally low rate of apoptotic cell death. The method is carried out by administering to the patient a compound that mediates oligomerization between Tango-63d and/or Tango-63e and intracellular polypeptides that interact with Tango-63d or Tango-63e to transduce an apoptotic signal that leads to the cell's death.

The invention also features a method of identifying a compound that modulates the activity of Tango-63d and/or Tango-63e by assessing the activity of Tango-63d and/or Tango-63e in the presence and absence of the compound.

In other aspects, the invention includes a method for determining whether a compound modulates oligomerization between Tango-63d and/or Tango-63e and polypeptides that form a complex with these polypeptides by examining oligomerization of Tango-63d and/or Tango-63e and these polypeptides in the presence and absence of the compound. An administered compound may modulate oligomerization between and Tango-63d or Tango-63e and, for example, Caspase-8 or Caspase-8-like polypeptides, TRADD or TRADD-like polypeptides, and FADD/MORT-1 or FADD-MORT-1-like polypeptides.

The invention features an isolated nucleic acid molecule that hybridizes under stringent conditions to a nucleic acid molecule having the nucleotide sequence of SEQ ID NO:1, the isolated nucleic acid molecule encoding Tango-63d; an isolated nucleic acid molecule that hybridizes under stringent conditions to a nucleic acid molecule having the nucleotide sequence of SEQ ID NO:3, the isolated nucleic acid molecule encoding Tango-63e; an isolated nucleic acid molecule that includes a nucleotide sequence that is at least 90% identical to the nucleotide sequence of SEQ ID NO:1, the isolated nucleic acid molecule encoding Tango-63d; and an isolated nucleic acid molecule that includes a nucleotide sequence which is at least 90% identical to the nucleotide sequence of SEQ ID NO:3, the isolated nucleic acid molecule encoding Tango-63e.

Also considered within the scope of the invention is a nucleic acid molecule that: hybridizes under stringent conditions to cDNA sequence contained within ATCC Accession No. 98367; hybridizes under stringent conditions to cDNA sequence contained within ATCC Accession No. 98368; is 85% identical to SEQ ID NO:1 (FIG. 1); is 85% identical to SEQ ID NO:3 (FIG. 2); is 95% identical to SEQ ID NO:1; is 95% identical to SEQ ID NO:3; is 85% identical to cDNA sequence contained within ATCC Accession No. 98367; is 85% identical to cDNA sequence contained within ATCC Accession No. 98368; is 95% identical to cDNA sequence contained within ATCC Accession No. 98367; is 95% identical to cDNA sequence contained within ATCC Accession No. 98368; hybridizes under stringent conditions to nucleotides 128 to 1447 of SEQ ID NO:1 (FIG. 1); or hybridizes under stringent conditions to nucleotides 128 to 1360 of SEQ ID NO:3 (FIG. 2). Polypeptides encoded by these nucleic acids are also considered within the scope of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the detailed description and from the claims. Although materials and methods similar or equivalent to those described herein can be used in the practice or testing of the invention, the preferred materials and methods are described below.

### BRIEF DESCRIPTION OF THE DRAWINGS

FIG. 1 is a representation of the nucleic acid sequence of Tango-63d (SEQ ID NO:1 (bottom)) and the amino acid sequence of the polypeptide it encodes (SEQ ID NO:2 (top)).

FIG. 2 is a representation of the nucleic acid sequence of Tango-63e (SEQ ID NO:3 (bottom)) and the amino acid sequence of the polypeptide it encodes (SEQ ID NO:4 (top)).

### DETAILED DESCRIPTION

The present invention relates to the discovery, identification, and characterization of two nucleic acid molecules that encode novel polypeptides, i.e., Tango-63d and Tango-63e.

### Nucleic Acid Molecules of the Invention

Isolated nucleic acid molecules, as defined above, can be cDNA, genomic DNA, synthetic DNA, or RNA, and can be double-stranded or single-stranded (i.e., either a sense or an antisense strand). Fragments of these molecules, which are also considered within the scope of the invention, can be produced, for example, by the polymerase chain reaction (PCR) or generated by treatment with one or more restriction endonucleases. A ribonucleic acid (RNA) molecule can be produced by in vitro transcription.

The nucleic acid molecules of the invention can contain naturally occurring sequences, or sequences that differ from those that occur naturally, but, due to the degeneracy of the genetic code, encode the same polypeptide. In addition, these nucleic acid molecules are not limited to sequences that only encode functional polypeptides, and thus, can include coding sequence that encodes a nonfunctional polypeptide, as well as some or all of the non-coding sequences that lie upstream or downstream from a coding sequence.

The nucleic acid molecules of the invention can be synthesized (for example, by phosphoramidite-based synthesis) or obtained from a biological cell, such as the cell of a mammal. Thus, the nucleic acids can be those of a human, mouse, rat, guinea pig, cow, sheep, horse, pig, rabbit, monkey, dog, or cat. Combinations or modifications of the nucleotides within these types of nucleic acids are also encompassed.

The isolated nucleic acid molecules of the invention encompass fragments that are not found as such in the natural state. Thus, the invention encompasses recombinant molecules, such as those in which a nucleic acid sequence (for example, a sequence encoding Tango-63d or Tango-63e) is incorporated into a vector (for example, a plasmid or viral vector) or into the genome of a heterologous cell (or the genome of a homologous cell, at a position other than the natural chromosomal location). These circumstances are discussed further below.

In the event the nucleic acid molecules of the invention encode or act as antisense molecules, they can be used for example, to regulate transcription of the nucleic acid molecules of the invention. With respect to regulation of Tango-63d or Tango-63e transcription, such techniques can be used to diagnose and/or treat disorders associated with apoptotic cell death. These nucleic acids will be discussed further in that context.

In addition to the nucleotide sequences disclosed herein (see, for example SEQ ID NOs:1 and 3), equivalent forms can be present in other species, and can be identified and isolated by using the nucleotide sequences disclosed herein and standard molecular biological techniques. For example, homologs of Tango-63d and Tango-63e can be isolated from other organisms by performing PCR using two degenerate oligonucleotide primer pools designed on the basis of amino acid sequences that are conserved in Tango-63d and Tango-63e. Alternatively, the method used to identify human Tango-63d and Tango-63e can be used to isolate homologs from other species. The template for the reaction can be cDNA obtained by reverse transcription of mRNA prepared from, for example, human or non-human cell lines or tissues, particularly those known or suspected to express Tango-63d or Tango-63e (see the expression data presented in the example below). The PCR product can be subcloned and sequenced to ensure that the amplified nucleic acid sequence represents the sequence of Tango-63d or Tango-63e. Once identified, Tango-63d and Tango-63e in other species can be used, in turn, to develop animal models for the purpose of drug discovery. Alternatively, these members of the TNF receptor superfamily can be used in in vitro assays for the purpose of drug discovery.

The invention also encompasses nucleotide sequences that encode mutant Tango-63d or Tango-63e, or fragments thereof, that retain one or more functions of Tango-63d or Tango-63e, as described herein.

The invention also encompasses: (a) expression vectors that contain any of the foregoing Tango-63d or Tango-63e coding sequences and/or their complements (that is, “antisense” sequence); (b) expression vectors that contain Tango-63d or Tango-63e coding sequences operatively associated with a regulatory element that directs the expression of the coding sequences; (c) expression vectors containing Tango-63d or Tango-63e nucleic acid molecules and heterologous nucleic acid molecules, such as molecules encoding a reporter or marker; and (d) genetically engineered host cells that contain any of the foregoing expression vectors and thereby express the nucleic acid molecules of the invention in the host cell.

As used herein, regulatory elements include but are not limited to inducible and non-inducible promoters, enhancers, operators and other elements, which are known to those skilled in the art, that drive and regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Similarly, the nucleic acid can form part of a hybrid gene encoding additional polypeptide sequences (for example, sequences that function as a marker or reporter) that can be used, for example, to produce a fusion protein (as described further below). Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

The expression systems that can be used for purposes of the invention include but are not limited to microorganisms such as bacteria (for example, E. coli and B. subtilis) transformed with recombinant bacteriophage DNA, plasmid DNA or cosmid DNA expression vectors containing the nucleic acid molecules of the invention; yeast (for example, Saccharomyces and Pichia) transformed with recombinant yeast expression vectors containing the nucleic acid molecules of the invention (preferably containing the nucleic acid sequences of Tango-63d and/or Tango-63e); insect cell systems infected with recombinant virus expression vectors (for example, baculovirus) containing the nucleic acid molecules of the invention; plant cell systems infected with recombinant virus expression vectors (for example, cauliflower mosaic virus (CamV) and tobacco mosaic virus (TMV)) or transformed with recombinant plasmid expression vectors (for example, Ti plasmid) containing Tango-63d and/or Tango-63e nucleotide sequences; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors can be advantageously selected depending upon the use intended for the gene product being expressed. For example, when a large quantity of such a protein is to be produced, for the generation of pharmaceutical compositions of Tango-63d or Tango-63e polypeptides for raising antibodies to those polypeptides, vectors that are capable of directing the expression of high levels of fusion protein products that are readily purified can be desirable. Such vectors include, but are not limited to, the E. coli expression vector pUR278 (Ruther et al., EMBO J. 2:1791, 1983), in which the coding sequence of the insert can be ligated individually into the vector in frame with the lacZ coding region so that a fusion protein is produced; pIN vectors (Inouye and Inouye, Nucleic Acids Res. 13:3101-3109, 1985; Van Heeke and Schuster, J. Biol. Chem. 264:5503-5509, 1989); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The coding sequence of the insert can be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect S. frugiperda cells in which the inserted gene is expressed. (for example, see Smith et al. J. Virol. 46:584, 1983; Smith, U.S. Pat. No. 4,215,051).

In mammalian host cells, a number of viral-based expression systems can be utilized. In cases where an adenovirus is used as an expression vector, the nucleic acid molecule of the invention can be ligated to an adenovirus transcription/translation control complex, for example, the late promoter and tripartite leader sequence. This chimeric gene can then be inserted in the adenovirus genome by in vitro or in viva recombination. Insertion in a non-essential region of the viral genome (for example, region E1 or E3) will result in a recombinant virus that is viable and capable of expressing the polypeptide encoded by the nucleic acid molecule of the invention in infected hosts (for example, see Logan and Shenk, Proc. Natl. Acad. Sci. USA 81:3655-3659, 1984). Specific initiation signals can also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals can be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner et al., Methods in Enzymol. 153:516-544, 1987).

In addition, a host cell strain can be chosen which modulates the expression of the inserted sequences, or modifies and processes the gene product in the specific fashion desired. Such modifications (for example, glycosylation) and processing (for example, cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used.

For long-term, high-yield production of recombinant proteins, stable expression is preferred. For example, cell lines which stably express Tango-63d or Tango-63e sequences described above can be engineered. Rather than using expression vectors which contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which produce Tango-63d and/or Tango-63e. Such engineered cell lines can be particularly useful in screening and evaluation of compounds that affect the endogenous activity of the gene product.

A number of selection systems can be used, including but not limited to the herpes simplex virus thymidine kinase (Wigler, et al., Cell 11:223, 1977), hypoxanthine-guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., Proc. Natl. Acad. Sci. USA 77:3567, 1980; O'Hare, et al., Proc. Natl. Acad. Sci. USA 78:1527, 1981); gpt, which confers resistance to mycophenolic acid (Mulligan and Berg, Proc. Natl. Acad. Sci. USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984).

Alternatively, any fusion protein can be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88:8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

### Polypeptides of the Invention

The Tango-63d and Tango-63e polypeptides described herein and fragments, mutants, and truncated forms thereof, including fusion proteins, can be prepared for a variety of uses, including but not limited to the generation of antibodies, as reagents in diagnostic assays, for the identification of other cellular gene products involved in the regulation of apoptosis, as reagents in assays for screening for compounds that can be used in the treatment of disorders associated with apoptotic cell death, or abnormal activity of polypeptides in the TNF receptor superfamily, and as pharmaceutical reagents useful in the treatment of such disorders.

The invention encompasses proteins and polypeptides that have one or more of the functions of naturally-occurring Tango-63d or Tango-63e. The functional attributes of Tango-63d and Tango-63e may include one or more of the following: the ability to bind TRADD, and the ability to initiate a biochemical reaction that induces apoptosis. Polypeptides having one or more functions of naturally-occurring Tango-63d or Tango-63e (i.e., functionally equivalent polypeptides) can include, but are not limited to, polypeptides that contain additions or substitutions of amino acid residues within sequences encoded by the nucleic acid molecules described above (see SEQ ID NOs:1 and 3), or that are encoded by nucleic acid molecules which result in a silent change, and thus produce a functionally equivalent gene product. Amino acid substitutions can be made on the basis of similarity in polarity, charge, solubility, hydrophobicity, hydrophilicity, and/or the amphipathic nature of the residues involved. Amino acids that are typically considered as providing a conservative substitution for one another are specified in the summary of the invention.

Random mutations can be made to Tango-63d or Tango-63e DNA using random mutagenesis techniques well known to those skilled in the art, and the resulting mutant polypeptides tested for activity. Alternatively, site-directed mutations can be engineered using site-directed mutagenesis techniques well known to those skilled in the art. The mutant polypeptides generated can have either an increased ability to function in lieu of Tango-63d or Tango-63e, for example, they can have a higher binding affinity for putative extracellular ligands or for intracellular polypeptides with which Tango-63d or Tango-63e may interact to form a complex that instigates apoptosis.

While the polypeptides of the invention can be chemically synthesized (for example, see Creighton, “Proteins: Structures and Molecular Principles,” W.H. Freeman & Co., NY, 1983), large polypeptides, i.e., polypeptides equivalent in size to Tango-63d or Tango-63e, can advantageously be produced by recombinant DNA technology including in vitro recombinant DNA techniques, synthetic techniques, and in vivo genetic recombination described herein. In addition, skilled artisans can consult Ausubel et al. (“Current Protocols in Molecular Biology, Vol. I,” Green Publishing Associates, Inc., and John Wiley & sons, Inc., NY, 1989), Sambrook et al. (“Molecular Cloning, A Laboratory Manual,” Cold Spring Harbor Press, Cold Spring Harbor, N.Y., 1989), and, particularly for examples of chemical synthesis, Gait, M. J. (Ed. “Oligonucleotide Synthesis,” IRL Press, Oxford, 1984), which are incorporated by reference herein in their entirety.

Also encompassed by the invention are polypeptides encoded by nucleic acid molecules which hybridize under stringent conditions to a nucleic acid molecule having the sequence of SEQ ID NO: 1; polypeptides encoded by nucleic acid molecules which hybridize under stringent conditions to a nucleic acid molecule having the sequence of SEQ ID NO:3; polypeptides encoded by nucleic acid molecules which hybridize under stringent conditions to a nucleic acid molecule having the sequence of the Tango-63d encoding portion of the clone designated ATCC accession number 98368; and polypeptides encoded by nucleic acid molecules which hybridize under stringent conditions to a nucleic acid molecule having the sequence of the Tango-63e encoding portion of the clone designated ATCC accession number 98367.

Antibodies

The invention also encompasses antibodies that bind Tango-63d or Tango-63e. Antibodies that specifically recognize one or more epitopes of these proteins, or fragments thereof are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention can be used, for example, in the detection of various forms of Tango-63d or Tango-63e in a biological sample and can, therefore, be utilized as part of a diagnostic or prognostic technique whereby patients can be tested for abnormal amounts of Tango-63d or Tango-63e. Such antibodies can also be utilized in conjunction with, for example, compound screening schemes, as described below, for the evaluation of the effect of test compounds on expression and/or activity of Tango-63d or Tango-63e. Additionally, such antibodies can be used in conjunction with the gene therapy techniques described below, to, for example, evaluate cells expressing the alternate forms described herein prior to their introduction into the patient. Preferably, the antibodies recognize epitopes of Tango-63d or Tango-63e that are unique, i.e., are not present on related molecules, such as members of the TNF receptor superfamily (e.g., TNFR-1) or more distantly related proteins. Accordingly, the antibodies are preferably raised against a peptide sequence present in Tango-63d or Tango-63e that is not present in related molecules, such as members of the TNF receptor superfamily.

For the production of antibodies, various host animals can be immunized by injection with a peptide having a sequence that is present in Tango-63d and/or Tango-63e. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil age emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, can be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B cell hybridoma technique (Kosbor et al., Immunology Today 4:72, 1983;Cole et al., Proc. Natl. Acad. Sci. USA 80:2026-2030, 1983), and the EBV-hybridoma technique (Cole et al., “Monoclonal Antibodies And Cancer Therapy,” Alan R. Liss, Inc., pp. 77-96, 1985). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA, 81:6851-6855, 1984; Neuberger et al., Nature, 312:604-608, 1984; Takeda et al., Nature, 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-426, 1988; Huston et al., Proc. Natl. Acad. Sci. USA 85:5879-5883, 1988; and Ward et al., Nature 334:544-546, 1989) can be adapted to produce single chain antibodies against Tango-63d or Tango-63e gene products. Single chain antibodies are formed by linking the heavy and light chain fragments of the Fv region via an amino acid bridge, resulting in a single chain polypeptide.

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., Science, 246:1275-1281, 1989) to allow rapid and easy identification of monoclonal Fab fragments with the desired specificity.

These antibodies can, in turn, be utilized to generate anti-idiotype antibodies that “mimic”Tango-63d or Tango-63e, using techniques well known to those skilled in the art. (See, for example, Greenspan and Bona, FASEB J. 7:437-444, 1993; and Nissinoff, J. Immunol. 147:2429-2438, 1991). Such neutralizing anti-idiotypes or Fab fragments of such anti-idiotypes can be used in diagnostic regimens to detect disorders associated with apoptotic cell death.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

The methods described herein can be performed, for example, by utilizing pre-packaged diagnostic kits comprising at least one specific Tango-63d or Tango-63e nucleotide sequence or antibody reagent described herein, which can be conveniently used,for example, in clinical settings, to diagnose patients exhibiting symptoms of the disorders described below.

Transgenic Animals

In another embodiment, the present invention relates to non-human, transgenic animals having cells that express the nucleic acid molecules of the invention. Preferably, the animals express Tango-63d and/or Tango-63e (e.g., encoded by a gene which produces Tango-63d or Tango-63e mRNA without splicing). Such transgenic animals represent a model system for the study of disorders that are caused by or exacerbated either by excessive or insufficient apoptotic cell death, and for the development of therapeutic agents that modulate the expression or activity of the polypeptides described herein. As defined above, animals such as mice, rats, rabbits, guinea pigs, pigs, micro-pigs, goats, and non-human primates, for example, baboons, monkeys, and chimpanzees can be used to generate these transgenic animals.

Preferably, the transgenic animals of the present invention are produced by introducing a nucleic acid molecule of the invention into single-celled embryos so that the DNA is stably integrated into the DNA of germ-line cells in the mature animal, and inherited in a Mendelian fashion. However, any technique known in the art can be used to introduce the transgene into animals to produce the founder lines of transgenic animals. Such techniques include, but are not limited to pronuclear microinjection (see, for example, U.S. Pat. No. 4,873,191); retrovirus mediated gene transfer into germ lines (Van der Putten et al., Proc. Natl. Acad. Sci., USA 82:6148-6152, 1985); gene targeting in embryonic stem cells (Thompson et al., Cell 56:313-321, 1989); electroporation of embryos (Lo, Mol Cell. Biol. 3:1803-1814, 1983); and sperm-mediated gene transfer (Lavitrano et al., 1989, Cell 57:717-723); etc. For a review of such techniques, see Gordon, 1989, Transgenic Animals, Intl. Rev. Cytol. 115:171-229. Skilled artisans can obtain additional guidance from, for example: Hogan et al. “Manipulating the Mouse Embryo” (Cold Spring Harbor Press, Cold Spring Harbor, N.Y., 1986; Krimpenfort et al., Bio/Technology 9:86, 1991; Palmiter et al., Cell 41:343, 1985; Kraemer et al., “Genetic Manipulation of the Early Mammalian Embryo” (Cold Spring Harbor Press, Cold Spring Harbor, N.Y., 1985; Hammer et al., Nature 315:680, 1985; Purcel et al., Science, 244:1281, 1986; Wagner et al., U.S. Pat. No. 5,175,385; and Krimpenfort et al., U.S. Pat. No. 5,175,384 (the latter two publications are hereby incorporated by reference).

The present invention provides for transgenic animals that carry the Tango-63-related transgene of the invention in all their cells, as well as animals which carry the transgene in some, but not all their cells, that is, the invention provides for mosaic animals. The transgene can be integrated as a single transgene or in concatamers, for example, head-to-head tandems or head-to-tail tandems. The transgene can also be selectively introduced into and activated in a particular cell type by following, for example, the teaching of Lasko et al. (Proc. Natl. Acad. Sci. USA 89:6232-6236, 1992). The regulatory sequences required for such a cell-type specific activation will depend upon the particular cell type of interest, and will be apparent to those of skill in the art.

When it is desired that the Tango-63d or Tango-63e transgene be integrated into the chromosomal site of the endogenous gene, gene targeting is preferred. Briefly, when such a technique is to be utilized, vectors containing some nucleotide sequences homologous to the endogenous Tango-63d or Tango-63e genes are designed for the purpose of integrating, via homologous recombination with chromosomal sequences, into and disrupting the function of the nucleotide sequence of the endogenous Tango-63 gene. A transgene can also be selectively introduced into a particular cell type, thus inactivating or “knocking out” the endogenous gene in only that cell type, by following, for example, the teaching of Gu et al. (Science 265:103-106, 1994). The regulatory sequences required for such a cell-type specific inactivation will depend upon the particular cell type of interest, and will be apparent to those of skill in the art.

The level of mRNA expression of the transgene in the tissues of the transgenic animals can be assessed using techniques which include but are not limited to Northern blot or RNAse protection analysis of tissue samples obtained from the animal.

Use of the Nucleic Acids, Polypeptides, and Antibodies of the Invention in the Diagnosis and Treatment of Disorders associated with Apoptotic Cell Death

As described herein, the nucleic acids, polypeptides, antibodies, and other reagents of the invention can be used in the diagnosis and treatment of disorders associated with apoptotic cell death. In general, disorders associated with decreased cell death are those in which the expression or activity of Tango-63d and/or Tango-63e can be insufficient. Thus, these disorders can be treated by enhancing the expression or activity of Tango-63d and/or Tango-63e. Conversely, disorders associated with increased cell death are those in which expression or activity of Tango-63d and/or Tango-63e is excessive, and which would respond to treatment regimes in which expression or activity of these genes is inhibited. The disorders amenable to treatment will first be briefly reviewed and a discussion of therapeutic applications will follow (see, for example, “Formulations and Use”).

In addition to the examples provided herein, skilled artisans can consult Thompson (Science 267:1456-1462, 1995) for additional discussion of the disorders associated with apoptotic cell death.

Whether a Disorder is Mediated by the Expression of Tango-63d or Tango-63e

If one can determine whether a disorder is associated with apoptotic cell death, and whether that cell death is influenced by expression of the polypeptides disclosed herein, it should be possible to determine whether that disorder can be diagnosed or treated with the nucleic acid, polypeptide, or antibody molecules of the invention. A disorder in which there is either insufficient or excessive cell death can be studied by determining whether Tango-63d or Tango-63e are either overexpressed or underexpressed in the affected tissue. The expression levels can be compared from tissue to tissue within a single patient, or between tissue samples obtained from a patient that is ill and one or more patients who are well. If it is determined that either Tango-63d, Tango-63e, or both are either overexpressed or underexpressed, it can be said that the disorder should be amenable to one or more of the treatment methods disclosed herein.

Diagnostic methods in which Tango-63d and Tango-63e are detected in a biological sample can be carried out, for example, by amplifying the nucleic acid molecules within the sample by PCR (the experimental embodiment set forth in Mullis, K. B., 1987, U.S. Pat. No. 4,683,202), followed by the detection of the amplified molecules using techniques well known to those of skill in the art. For example, for detection of the amplified product, the nucleic acid amplification can be performed using radioactively or non-radioactively labeled nucleotides. Alternatively, enough amplified product can be made such that the product can be visualized by standard ethidium bromide staining or by utilizing any other suitable nucleic acid staining method. The resulting amplified sequences can be compared to those which were obtained either from a tissue that is not affected by the disorder, from a person who is well, or that were obtained from the patient before the disorder developed.

The level of expression of Tango-63d and Tango-63e can also be assayed by detecting and measuring transcription. For example, RNA from a cell type or tissue that is known, or suspected to express these polypeptides, can be isolated and tested utilizing the PCR techniques described above.

The analysis of cells taken from culture can be a necessary step in the assessment of cells to be used as part of a cell-based-gene therapy technique or, alternatively, to test the effect of compounds on the expression of Tango-63d and Tango-63e. Such analyses can reveal both quantitative and qualitative aspects of the expression pattern of the polypeptides of the invention, including activation or inactivation of their expression.

Where a sufficient quantity of the appropriate cells can be obtained, standard Northern blot or RNAse protection analyses can be performed to determine the level of mRNA encoding polypeptides of the invention, particularly Tango-63d and Tango-63e.

It is also possible to base diagnostic assays and screening assays for therapeutic compounds on detection of Tango-63d polypeptide or Tango-63e polypeptide. Such assays for Tango-63d polypeptide or Tango-63e polypeptide, or peptide fragments thereof will typically involve incubating a sample, such as a biological fluid, a tissue extract, freshly harvested cells, or lysates of cells which have been incubated in cell culture, in the presence of a detectably labeled antibody capable of identifying these gene products (or peptide fragments thereof), and detecting the bound antibody by any of a number of techniques well-known in the art.

The biological sample can be brought in contact with and immobilized onto a solid phase support or carrier such as nitrocellulose, or other solid support which is capable of immobilizing cells, cell particles, or soluble proteins. The support can then be washed with suitable buffers followed by treatment with the detectably labeled antibody or fusion protein. The solid phase support can then be washed with the buffer a second time to remove unbound antibody or fusion protein. The amount of bound label on solid support can then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-Tango-63d or anti-Tango-63e antibody or fusion proteins containing these polypeptides can be determined according to well known methods. Those skilled in the art will be able to determine operative and optimal assay conditions for each determination by employing routine experimentation.

With respect to antibodies, one of the ways in which the antibody of the instant invention can be detectably labeled is by linking it to an enzyme for use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.; Voller et al., J. Clin. Pathol. 31:507-520, 1978; Butler, Meth. Enzymol. 73:482-523, 1981; Maggio, E. (ed.), “Enzyme Immunoassay,” CRC Press, Boca Raton, Fla., 1980; Ishikawa, E. et al., (eds.), “Enzyme Immunoassay,” Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibodies or antibody fragments, it is possible to detect Tango-63d and Tango-63e through the use of a radioimmunoassay (RIA) (see, for example, Weintraub, B., “Principles of Radioimmunoassays, Seventh Training Course on Radioligand Assay Techniques,” The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Still further, the invention encompasses methods and compositions for the treatment of the disorders described above, and any others that are found to be associated with apoptotic cell death. Such methods and compositions are capable of modulating the level of expression of Tango-63d or Tango-63e and/or the level of activity of the gene products.

Numerous ways of altering the expression or activity of the polypeptides of the invention are known to skilled artisans. For example, living cells can be transfected in vivo with the nucleic acid molecules of the invention (or transfected in vitro and subsequently administered to the patient). For example, cells can be transfected with plasmid vectors by standard methods including, but not limited to, liposome- polybrene-, or DEAE dextran-mediated transfection (see, e.g., Felgner et al., Proc. Natl. Acad. Sci. USA 84:7413, 1987; Ono et al., Neurosci. Lett. 117:259, 1990; Brigham et al., Am. J. Med. Sci. 298:278, 1989), electroporation (Neumann et al., EMBO J. 7:841, 1980), calcium phosphate precipitation (Graham et al., Virology 52:456, 1973; Wigler et al., Cell 14:725, 1978; Felgner et al., supra) microinjection (Wolff et al., Science 247:1465, 1990), or velocity driven microprojectiles (“biolistics”).

These methods can be employed to mediate therapeutic application of the molecules of the invention. For example, antisense nucleic acid therapies or ribozyme approaches can be used to inhibit utilization of Tango-63d and/or Tango-63e mRNA; triple helix approaches can also be successful in inhibiting transcription of various polypeptides in the TNF receptor superfamily. Antisense approaches involve the design of oligonucleotides (either DNA or RNA) that are complementary to the mRNA molecules of the invention. The antisense oligonucleotides will bind to the complementary mRNA transcripts and prevent translation. Antisense oligonucleotides must be specific for the mRNA of interest. Accordingly, oligonucleotides disclosed herein as SEQ ID NOs:8, 9, 10, and 11 are especially preferred. For example, the following oligonucleotides are suitable for specifically binding Tango-63d or Tango-63e mRNA:5′-CATGGCGGTAGGGAACGCTCT-3′ (SEQ ID NO:8; the reverse and complement of nucleotides 128-148),5′-GTTCTGTCCCCGTTGTTCCAT-3′ (SEQ ID NO:9; the reverse and complement of nucleotides 110-130). The following oligonucleotides are suitable for specifically binding Tango-63d mRNA because they bind to sequences that are not present in Tango-63e: 5′-GGCTTCCCCACTGTGCTTTGT-3′(SEQ ID NO:10); and 5′-GGAGGTCACCGTCTCCTCCAC-3′ (SEQ ID NO:11).

Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA can thus be tested, or triplex formation can be assayed. The ability to hybridize will depend on both the degree of complementarity and the length of the antisense nucleic acid. Generally, the longer the hybridizing nucleic acid, the more base mismatches with an RNA it can contain and still form a stable duplex (or triplex, as the case may be). One skilled in the art can ascertain a tolerable degree of mismatch by use of standard procedures to determine the melting point of the hybridized complex. Antisense oligonucleotides complementary to mRNA coding regions are less efficient inhibitors of translation than oligonucleotides that are complementary to 5′- or 3′-untranslated sequence, but could be used in accordance with the instant invention. The antisense nucleic acids should be at least six nucleotides in length, and are preferably oligonucleotides ranging from 6 to about 50 nucleotides in length. In specific aspects, the oligonucleotide is at least 10 nucleotides, preferably at least 17 nucleotides, more preferably at least 25 nucleotides, or most preferably at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using an antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotide can be modified at the base moiety, sugar moiety, or phosphate backbone, for example, to improve stability of the molecule, hybridization, etc. The oligonucleotide can include other appended groups such as peptides (for example, for targeting host cell receptors in vivo), or agents facilitating transport across the cell membrane (see, for example, Letsinger et al., Proc. Natl. Acad. Sci. USA 86:6553-6556, 1989; Lemaitre et al., Proc. Natl. Acad. Sci. USA 84:648-652, 1987; PCT Publication No. WO88/09810, published Dec. 15, 1988) or the blood-brain barrier (see, for example, PCT Publication No. WO89/10134, published Apr. 25, 1988), hybridization-triggered cleavage agents. (See, for example, Krol et al., BioTechniques 6:958-976, 1988) or intercalating agents (see, for example, Zon, Pharm. Res. 5:539-549, 1988). To this end, the oligonucleotide can be conjugated to another molecule, for example, a peptide, hybridization triggered cross-linking agent, transport agent, hybridization-triggered cleavage agent, and the like.

The antisense oligonucleotide can comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide can also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). The oligonucleotide is a 2′-O-methylribonucleotide (Inoue et al., Nucl. Acids Res. 15:6131-6148, 1987), or a chimeric RNA-DNA analogue (Inoue et al., FEBS Lett. 215:327-330, 1987).

Oligonucleotides of the invention can be synthesized by standard methods known in the art, for example, by use of an automated DNA synthesizer (such as are commercially available from Biosearch, Applied Biosystems, etc.). As examples, phosphorothioate oligonucleotides can be synthesized by the method of Stein et al. (Nucl. Acids Res. 16:3209, 1988), methylphosphonate oligonucleotides can be prepared by use of controlled pore glass polymer supports (Sarin et al., Proc. Natl. Acad. Sci. USA 85:7448-7451, 1988), etc.

The antisense molecules should be delivered to cells which express Tango-63d and/or Tango-63e in vivo. A number of methods have been developed for delivering antisense DNA or RNA to cells; for example, antisense molecules can be injected directly into the tissue site, or modified antisense molecules, designed to target the desired cells (for example, antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous Tango-63d and/or Tango-63e transcripts and thereby prevent translation of the Tango-63d and/or Tango-63e mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, Nature 290:304-310, 1981), the promoter contained in the 3′ long terminal repeat of Rous sarcoma virus (Yamamoto et al., Cell 22:787-797, 1980), the herpes thymidine kinase promoter (Wagner et al., Proc. Natl. Acad. Sci. USA 78:1441-1445, 1981), the regulatory sequences of the metallothionein gene (Brinster et al., Nature 296:39-42, 1982), and so forth. Any type of plasmid, cosmid, YAC or viral vector can be used to prepare the recombinant DNA construct which can be introduced directly into the tissue site; for example, the choroid plexus or hypothalamus. Alternatively, viral vectors can be used which selectively infect the desired tissue; (for example, for brain, herpesvirus vectors can be used), in which case administration can be accomplished by another route (for example, systemically).

Methods of designing antisense nucleic acids and introducing them into host cells have been described in, for example, Weinberg et al. (U.S. Pat. No. 4,740,463; hereby incorporated by reference).

Alternatively, the nucleic acid molecules of the invention can be administered so that expression of the Tango-63d and/or Tango-63e occurs in tissues where it does not normally occur, or is enhanced in tissues where it is normally expressed. This application can be used, for example, to suppress apoptotic cell death and thereby treat disorders in which cellular populations are diminished, such as those described herein as “disorders associated with diminished cell survival.” Preferably, the therapeutic nucleic acid (or recombinant nucleic acid construct) is applied to the site where cells are at risk of dying by apoptosis, to the tissue in the larger vicinity, or to the blood vessels supplying these areas.

Ideally, the production of a polypeptide that is a form of Tango-63d or Tango-63e (including forms that are involved in mediating apoptosis) by any gene therapy approach described herein, will result in a cellular level of expression that is at least equivalent to the normal, cellular level of expression of Tango-63d or Tango-63e. Skilled artisans will recognize that these therapies can be used in combination with more traditional therapies, such as surgery, radiotherapy, or chemotherapy. Accordingly, and as described below, the invention features therapeutic compositions that contain the nucleic acid molecules, polypeptides, and antibodies of the invention, as well as compounds that are discovered, as described below, to affect them.

Therapeutic Compositions

The nucleic acid molecules encoding Tango-63d and Tango-63e, the polypeptides themselves, antibodies that specifically bind Tango-63d and/or Tango-63e and compounds that affect the expression or activity of Tango-63d or Tango-63e can be administered to a patient at therapeutically effective doses to treat or ameliorate disorders associated with apoptotic cell death. A therapeutically effective dose refers to the dose that is sufficient to result in amelioration of symptoms of disorders associated with apoptotic cell death.

Effective Dose

Toxicity and therapeutic efficacy of a given compound can be determined by standard pharmaceutical procedures, using either cells in culture or experimental animals to determine the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce the danger or severe side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Levels in plasma can be measured, for example, by high performance liquid chromatography.

Formulations and Use

Pharmaceutical compositions for use in accordance with the present invention can be formulated in a conventional manner using one or more physiologically acceptable carriers or excipients.

Thus, the compounds and their physiologically acceptable salts and solvates can be formulated for administration by inhalation or insufflation (either through the mouth or the nose), or for oral, buccal, parenteral, or rectal administration.

For oral administration, the pharmaceutical compositions can take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl. sulphate). The tablets can be coated by methods well known in the art. Liquid preparations for oral administration can take the form of, for example, solutions, syrups or suspensions, or they can be presented as a dry product for constitution with water or another suitable vehicle before use. Such liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration, the compositions can take the form of tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, for example, gelatin for use in an inhaler or insufflator can be formulated containing a powder mix of the compound and a suitable powder base such as lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, for example, sterile pyrogen-free water, before use.

The compounds can also be formulated in rectal compositions such as suppositories or retention enemas, for example, containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

The therapeutic compositions of the invention can also contain a carrier or excipient, many of which are known to skilled artisans. Excipients which can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, (for example, intravenous, subcutaneous, intramuscular, intracranial, intraorbital, opthalmic, intraventricular, intracapsular, intraspinal, intracisternal, intraperitoneal, or transmucosal administration) or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.” It is expected that the preferred route of administration will be intravenous.

It is well known in the medical arts that dosages for any one patient depend on many factors, including the general health, sex, weight, body surface area, and age of the patient, as well as the particular compound to be administered, the time and route of administration, and other drugs being administered concurrently.

Dosages for the polypeptides and antibodies of the invention will vary, but a preferred dosage for intravenous administration is approximately 0.01 mg to 100 mg/ml blood volume. Determination of the correct dosage within a given therapeutic regime is well within the abilities of one of ordinary skill in the art of pharmacology. Skilled artisans will be aided in their determination of an adequate dosage by previous studies. For example, Abraham et al. (J. Amer. Med. Assoc. 273:934-941, 1995) administered TNF-α monoclonal antibody (TNF-α-MAb) at doses ranging from 1 to 15 mg/kg. The antibody was well tolerated by all patients, even though they developed human antimurine antibodies; no serum sickness-like reactions, adverse skin reactions, or systemic allergic reactions developed. Similarly, Rankin et al. (Br. J. Rheumatol. 34:334-342, 1995) administered a single intravenous dose of 0.1, 1.0, or 10 mg/kg of an engineered human antibody, CDP571, which neutralizes human TNF-α. Both studies describe in detail how to evaluate patients who have been treated with antibodies.

Identification of Compounds that Mediate Oligomerization Between Polypeptides Within a Tango-63d- or Tango-63e-containing Complex

It has been shown (see Background of the Invention) that apoptosis can be induced by the formation of specific complexes of polypeptides, for example those that assemble when TNFR-1 or the Fas receptor are bound. Given the conservation between the intracellular domains of TNFR-1, Tango-63d, and Tango-63e, the same or similar polypeptides may assemble with Tango-63d or Tango-63e. Therefore, apoptosis can be inhibited within a cell that contains compounds that specifically inhibit interaction between Tango-63d and/or Tango-63e and polypeptides that would otherwise assemble to form a complex with these polypeptides. Conversely, apoptosis can be stimulated within a cell containing compounds that specifically promote interaction between Tango-63d and/or Tango-63e and one or more additional polypeptides. Accordingly, the invention features a method for treating a patient who has a disorder associated with an abnormally high rate of apoptotic cell death by administering to the patient a compound that inhibits oligomerization between Tango-63d or Tango-63e and other polypeptides. Patients who suffer instead from an abnormally low rate of apoptotic cell death can be treated with a compound that promotes oligomerization between these polypeptides.

The invention also features methods for screening compounds to identify those which increase or decrease the interaction between either Tango-63d and Tango-63e and other polypeptides. One suitable assay for determining whether another polypeptide has become associated with Tango-63d or Tango-63e is an immunoprecipitation assay. A suitable immunoprecipitation assay is described by Kischkel et al. (EMBO J. 14:5579, 1995). Anti-Tango-63d or Anti-Tango-63e antibodies can be used to perform these assays in the presence and absence of selected compounds, and to thereby identify those that increase or decrease association between polypeptides within the Tango-63d and Tango-63e complexes.

Recently, compounds that can penetrate the cell membrane were devised and shown to be capable of controlling the intracellular oligomerization of specific proteins. More specifically, ligands were used to induce intracellular oligomerization of cell surface receptors that lacked their transmembrane and extracellular regions but that contained intracellular signaling domains. Spencer et al. (Science 262:1019-1024, 1993) reported that addition of these ligands to cells in culture resulted in signal transmission and specific target gene activation. Further, these investigators proposed the use of these ligands “wherever precise control of a signal transduction pathway is desired. ” For further guidance in the use of synthetic ligands to induce dimerization of proteins, see Belshaw et al. (Proc. Natl. Acad. Sci. USA 93:4604-4607). This approach can be used to induce intracellular oligomerization within a Tango-63d- or Tango-63e-containing complex.

Identification of Compounds that Modulate the Expression or Activity of Tango-63d or Tango-63e

Isolation of the nucleic acid molecules described above (i.e. those encoding Tango-63d and Tango-63e) also facilitates the identification of compounds that can increase or decrease the expression of these molecules in vivo. To discover such compounds, cells that express Tango-63d and/or Tango-63e are cultured, exposed to a test compound (or a mixture of test compounds), and the level of Tango-63d and/or Tango-63e expression or activity is compared with the level of expression or activity in cells that are otherwise identical but that have not been exposed to the test compound(s). Many standard quantitative assays of gene expression can be utilized in this aspect of the invention. Examples of these assays are provided below.

In order to identify compounds that modulate expression of Tango-63d or Tango-63e (or homologous genes), the candidate compound(s) can be added at varying concentrations to the culture medium of cells that express Tango-63d or Tango-63e, as described above. These compounds can include small molecules, polypeptides, and nucleic acids. The expression of Tango-63d and Tango-63e is then measured, for example, by Northern blot, PCR analyses or RNAse protection analyses using a nucleic acid molecule of the invention as a probe. The level of expression of the polypeptides of the invention in the presence of the candidate molecule, compared with their level of expression in its absence, will indicate whether or not the candidate molecule alters the expression of Tango-63d, Tango-63e or other polypeptides of the invention.

Similarly, compounds that modulate the expression of the polypeptides of the invention can be identified by carrying out the assay described above and then performing a Western blot analysis using antibodies that bind Tango-63d or Tango-63e.

The test compounds, by altering the expression of Tango-63d or Tango-63e will, in turn, alter the likelihood that the cell in which these molecules are expressed will undergo apoptosis. For example, if the test compound decreases the expression of Tango-63d or Tango-63e, the cell will be less likely to undergo apoptosis. In contrast, if the test compound increases the expression of Tango-63d or Tango-63e, the cell will be more likely to under apoptosis. Thus, compounds identified in this way can be used as agents to control apoptosis and, in particular, as therapeutic agents for the treatment of various disorders associated with apoptosis (described above).

Compounds that alter the activity of Tango-63d or Tango-63e (e.g., by altering the affinity of these polypeptides for putative ligands or other compounds with which they may interact, or alternatively, by changing the fidelity with which they transduce an apoptotic signal) can be identified using the oligomerization and apoptosis assays described in detail above.

### EXAMPLE 1

### Identification and Characterization of Nucleic Acid Molecules Encoding Tango-63d and Tango-63e

Human prostate epithelial cells were obtained from Clonetics Corporation (San Diego, Calif.) and expanded in culture with Prostate Epithelial Growth Medium (PrEGM; Clonetics) according to the recommendations of the supplier. When the cells reached 80% confluence, they were cultured in Prostate Basal Media (Clonetics) for 24 hours. The prostate cells were then stimulated with PrEGM and cycloheximide (CHI; 40 μg/ml) for 3 hours. Total RNA was isolated using the RNeasy™ Midi Kit (Qiagen; Chatsworth, Calif.), and the polyA⁺ fraction was further purified using Oligotex™ beads (Qiagen).

Three μg of polyA⁺ RNA were used to synthesize a cDNA library using the Superscript™ cDNA synthesis kit (Gibco BRL, Gaithersburg, Md.). Complementary DNA was directionally cloned into the expression plasmid pMET7 using the SalI and NotI sites in the polylinker to construct a plasmid library. Transformants were picked and grown up for single-pass sequencing. Additionally, prostate cDNA was ligated into the SalI/NotI sites of the ZipLox™ vector (Gibco BRL) for construction of a lambda phage cDNA library.

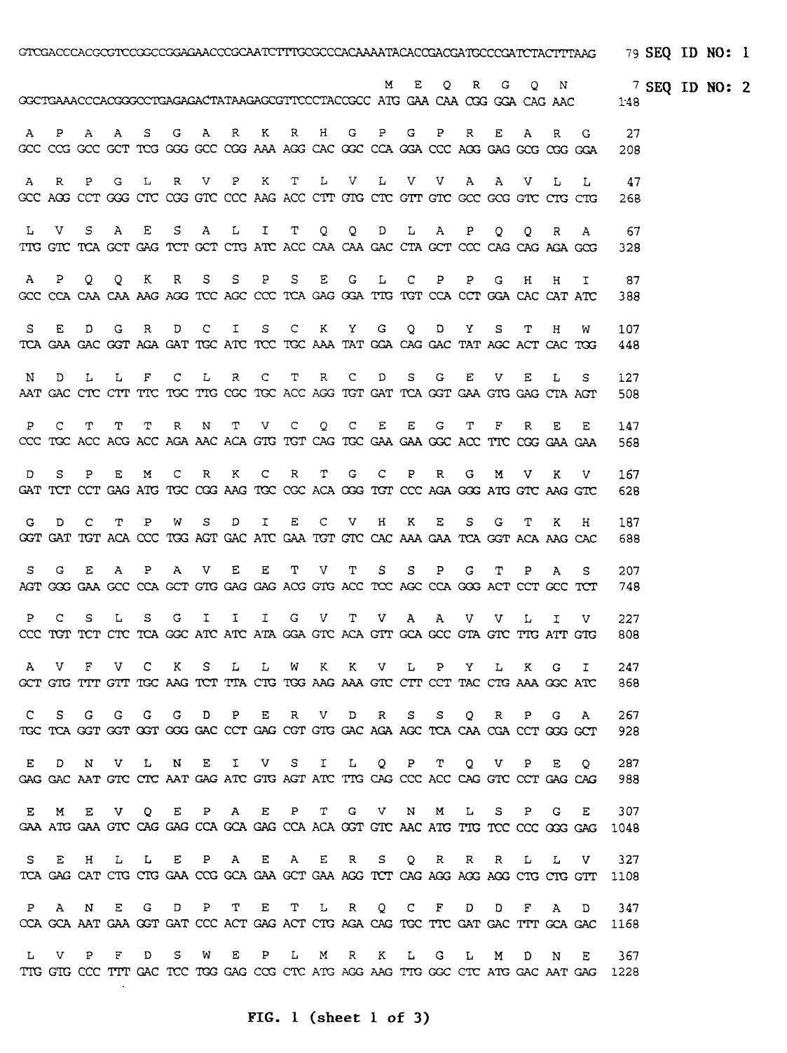

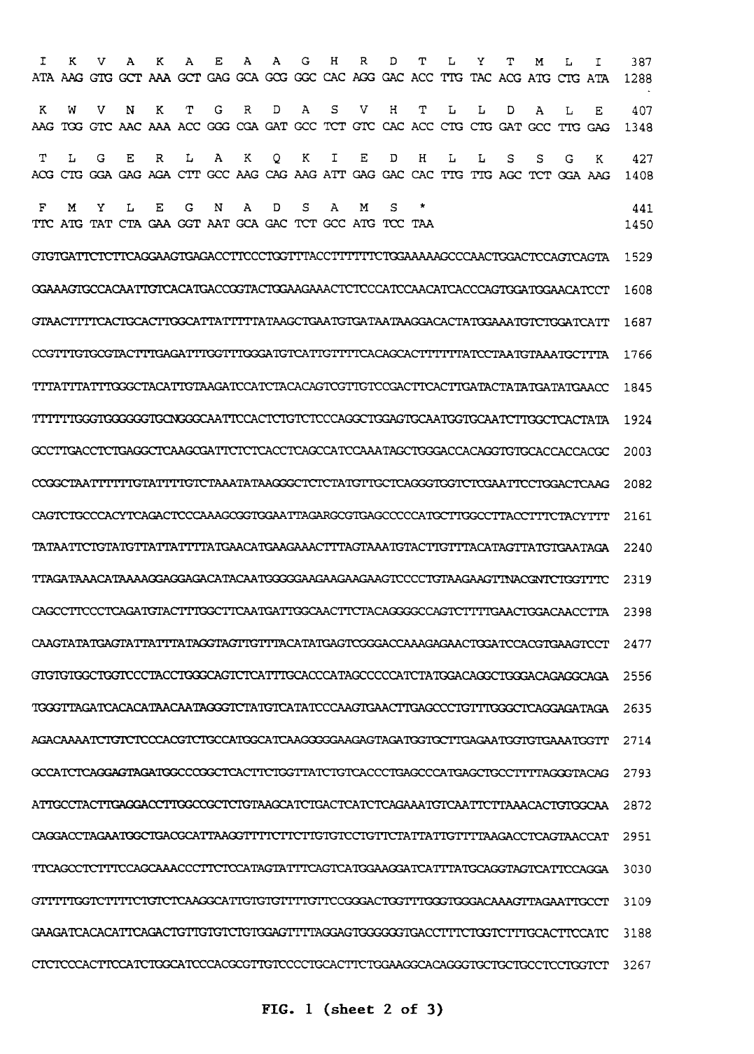

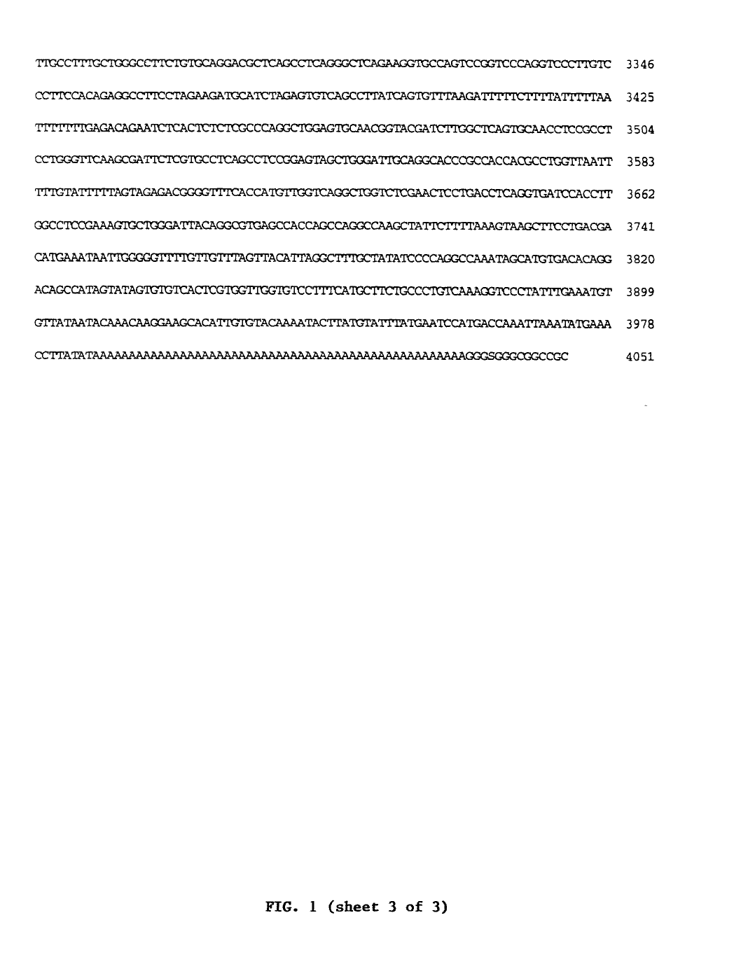

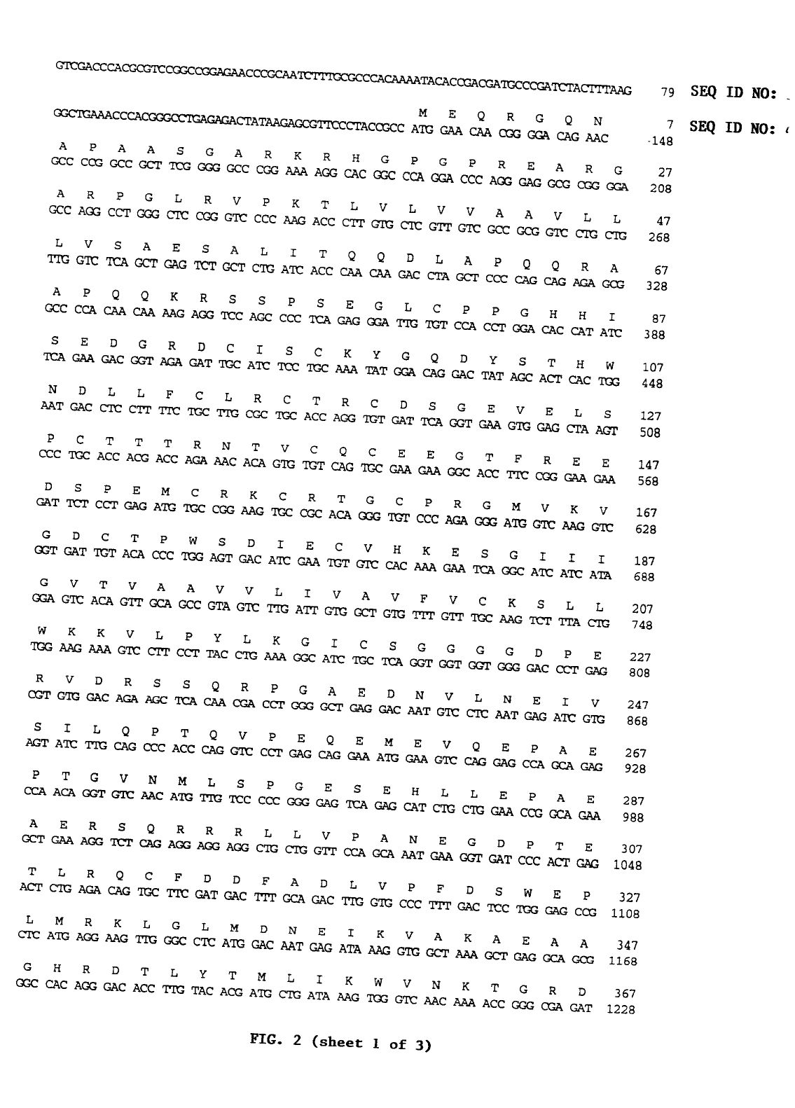

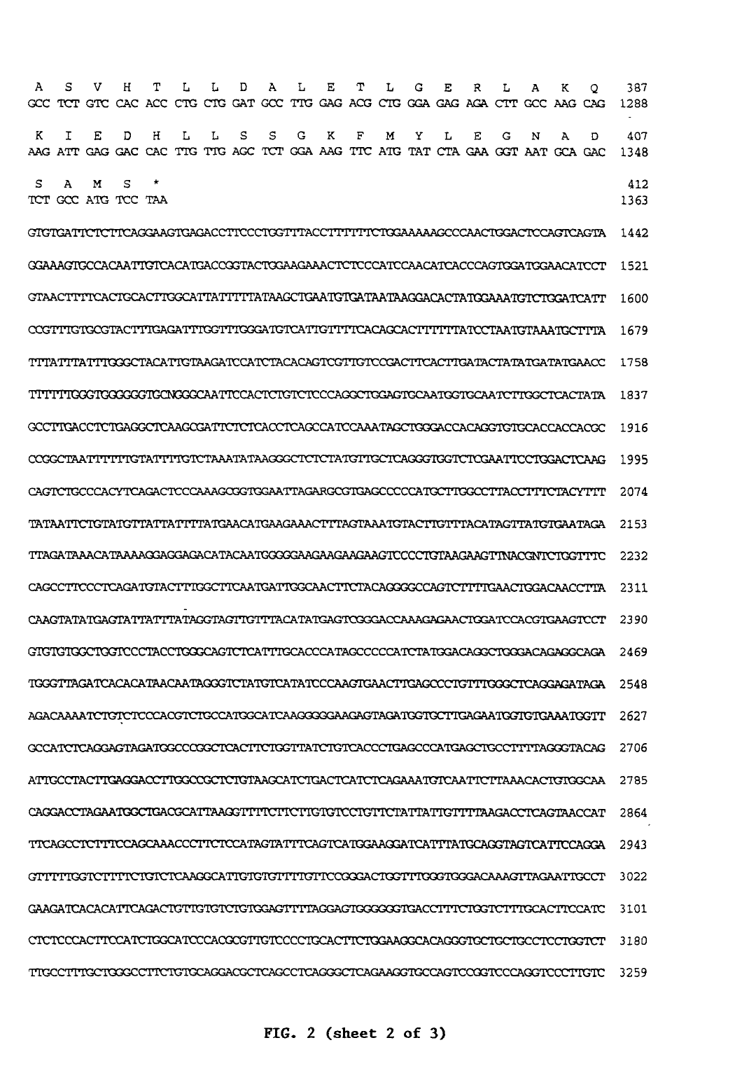

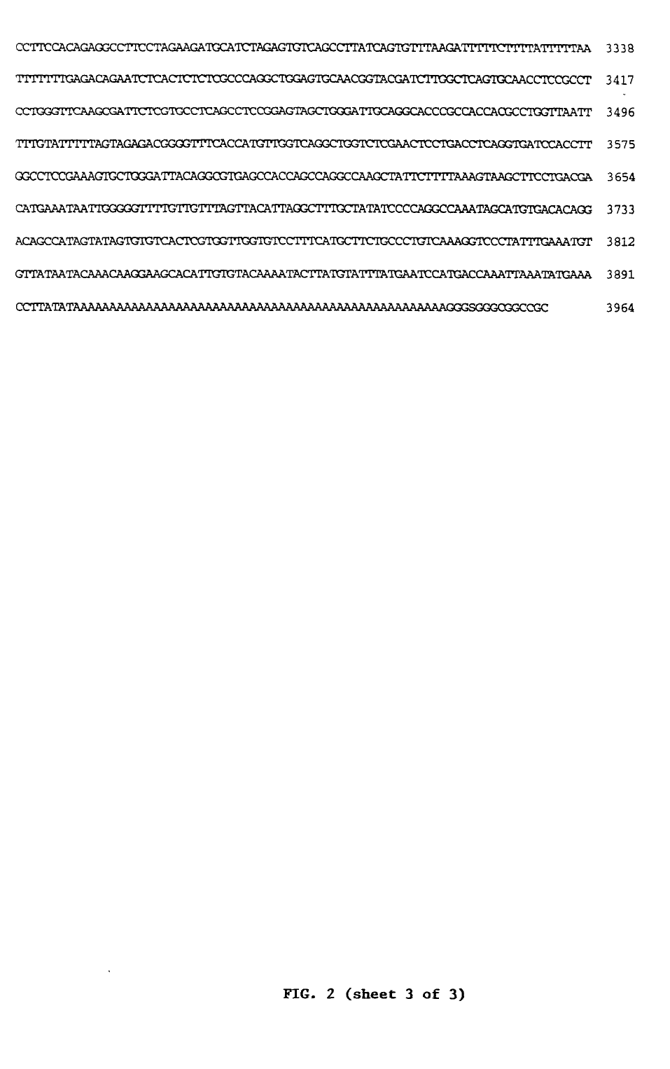

Two different forms of Tango-63 have been identified in the prostate cDNA library through EST sequencing and screening of the lambda phage library for the isolation of additional clones (Tango-63d and Tango-63e). Tango-63d encodes a polypeptide of 440 amino acids (encoded by nucleotides 128 to 1447 of SEQ ID NO: 1 and shown in FIG. 1); and Tango-63e encodes a polypeptide of 411 amino acids (encoded by nucleotides 128 to 1360 of SEQ ID NO: 3 nad shown in FIG. 2). The polypeptide encoded by Tango-63e is identical to that encoded by Tango-63d, with the exception of the deletion of amino acids 183-211 (encoded by nucleotides 677-760) in the Tango-63d sequence. The deleted amino acids are those just amino-terminal to the transmembrane domain in Tango-63d. Tango-63d and Tango-63e are novel polypeptides that represent new members of the tumor necrosis factor (TNF) receptor superfamily.

The members of the TNFR receptor superfamily are characterized by the presence of cysteine-rich repeats in their extracellular domains, and the Fas/APO-1 receptor and TNFR-1 also share an intracellular region of homology designated the “death domain” because it is required to signal apoptosis (Itoh and Nagata, J. Biol. Chem. 268:10932-10937, 1993). As described above, this shared death domain suggests that both receptors interact with a related set of signal-transducing molecules.

Tissue Distribution of Tango-63

The expression of Tango-63 (which is subsequently alternatively spliced to produce the novel polypeptides of the invention, Tango-63d and Tango-63e) was analyzed using Northern blot hybridization. A 422 base pair DNA fragment was generated using PCR with the following two oligonucleotides: LRH1 (5′-ATGGAACAACGGGGACAG-3′(SEQ ID NO:6); nucleotide positions 128-145 in Tango-63d) and LRH3 (5′-TTCTTCGCACTGACACAC-3′(SEQ ID NO:7); reverse and complement to nucleotide positions 533-550 in Tango-63d for use as a probe. The DNA was radioactively labeled with ³²P-dCTP using the Prime-It™ kit (Stratagene, La Jolla, Calif.) according to the instructions of the supplier. Filters containing human mRNA (MTNI and MTNII from Clontech, Palo Alto, Calif.) were probed in ExpressHyb™ hybridization solution (Clontech) and washed at high stringency. More specifically, the wash was carried out by submerging the filters in 2×SSC, 0.05% SDS at 55° C. (2×20 minutes) and then in 0.1×SSC, 0.1% SDS at 55° C. (2×20 minutes).

Tango-63 is expressed as a 4.2 kilobase (kb) transcript in a wide variety of human tissues including heart, placenta, lung, liver, skeletal muscle, kidney, pancreas, spleen, thymus, prostate, testis, ovaries, small intestine, colon, and peripheral blood leukocytes. Expression of Tango-63 was also detectable in the brain, but at significantly lower levels than in other tissues. Additional, but fainter, bands at about 2.2 kb (liver) and 1.0 kb (skeletal muscle) were also observed. These bands could represent additional forms of Tango-63, degradation products, or cross-reacting mRNAs.

An Assay for Tango-63d and Tango-63e Mediated Apoptosis

An assay for Tango-63d- or Tango-63e-mediated apoptosis can be used in screening assays to identify compounds that increase or decrease the degree of apoptosis within a population of cells. The compounds identified using these assays can alter the degree of apoptosis by altering the expression of Tango-63d or Tango-63e, the activity of Tango-63d or Tango-63e, or the way in which these polypeptides interact with other polypeptides. Compounds identified in these assays can be used as therapeutic compounds to treat disorders associated with an abnormal rate of apoptosis.

Assays of apoptosis, particularly when apoptosis is mediated by a polypeptide in the TNF receptor superfamily, generally employ an antibody directed against the polypeptide, which, upon binding, initiates apoptosis. Alternatively, an assay that requires only overexpression of the polypeptide of interest can be performed. An example of such an assay is described below.

The activity of the polypeptides of the invention can be assayed via a cotransfection assay that is based on co-uptake (transfection) with plasmids that encode a polypeptide of the invention. The assay described below is based on the observation that overexpression of TNFR-1, DR-3, and several other death inducing molecules, such as Caspases, is sufficient to cause apoptosis in the absence of other stimuli. The assay described below demonstrates the ability of the novel polypeptides of the invention to diminish the number of cells surviving in culture by activating apoptosis.

β-galactosidase expression assays were performed essentially as described by Kumar et al. (Genes & Dev. 8:1613-1626, 1994). SW480 cells, derived from a human colon carcinoma, were cultured in Dulbecco's modified Eagle's medium (DMEM), high glucose, supplemented with 10% fetal calf serum and 100 μg/ml each of penicillin G and streptomycin. The cells were seeded at a density of 3×10⁵ cells/well on 6-well (35 mm) plates and grown in 5% CO₂ at 37° C. The following day, the cells were transfected with 0.5 μg of pSVβ (Clontech), which carries an insert encoding β-galactosidase, and 2.5 μg of either a control or an experimental plasmid using Lipofectamine™ reagent (Life Technologies) and Opti-MEM™ medium (Life Technologies). The experimental plasmids contained inserts encoding Tango-63d or Tango-63e; the control plasmids were otherwise identical except the Tango-63d or Tango-63e inserts were absent. Thirty-six hours following transfection, the cells were rinsed twice with phosphate-buffered saline (PBS), fixed, and stained for 6 hours or more at 37° C. If desired, the cells can remain in the staining solution at room temperature for longer periods of time. The staining process consisted of exposure to 1% X-gal, 4 mM potassium ferricyanide, and 2 mM magnesium chloride in PBS. After staining, the cells were examined with a light microscope for the appearance of blue color, indicating successful transfection.

The result of transfection with the control plasmid (encoding β-gal) and either the control or experimental plasmid (encoding Tango-63d or Tango-63e) was assessed by determining the percentage of blue (i.e. transfected) cells in each well or by counting the total number of blue cells in each well. In preliminary experiments, expression of Tango-63d or Tango-63e caused approximately 90% reduction in the number of βgal positive cells remaining in culture.

Numerous substances are capable of inducing apoptosis in various cell types and can thus be used in assays of apoptosis. These substances include physiological activators, such as TNF family members (for example, Fas ligand, TNFα, and TRAIL/APO2). Cell death can also be induced when growth factors are withdrawn from the medium in which the cells are cultured. Additional inducers of apoptosis include heat shock, viral infection, bacterial toxins, expression of the oncogenes myc, rel, and EIA, expression of tumor suppressor genes, cytolytic T cells, oxidants, free radicals, gamma and ultraviolet irradiation, β-amyloid peptide, ethanol, and chemotherapeutic agents such as Cisplatin, doxorubicin, arabinoside, nitrogen mustard, methotrexate, and vincristine.

### EXAMPLE 3

### Expression of Recombinant Tango-67 in COS Cells

A vector for expression of Tango-67 can be prepared using a vector pcDNAI/Amp (Invitrogen). This vector includes: a SV40 origin of replication, an ampicillin resistance gene, an E. coli replication origin, a CMV promoter followed by polylinker region, a SV40 intron, a and polyadenylation site. A DNA fragment encoding Tango-67 is cloned into the polylinker region of the vector such that Tango-67 expression is under the control of the CMV promoter. A DNA sequence encoding Tango-67 is prepared by PCR amplification of a Tango-67 using primers which include restriction sites that are compatible with the polylinker. The Tango-67 sequence is inserted into the vector. The resulting construct is used to transform E. coli strain SURE (Stratagene, La Jolla, Calif.) and amp resistant colonies are selected. Plasmid DNA is isolated from transformants and examined by restriction analysis the presence of the correct fragment. For expression of the recombinant Tango-67, COS cells are transfected with the expression vector by DEAE-DEXTRAN method and grown is standard tissue culture medium.

Chromosome 8p Loss of Heterozygosity (LOH) and Tango-63

In tumor tissues and cultured cancer cells, loss of heterozygosity (LOH) is much more frequently observed on the short arm of human chromosome 8p than on any other human chromosome. Tumor suppressor genes have been identified in regions of frequent LOH in tumor samples (e.g., p53, Rb, APC, DCC-DPC4). The frequency of LOH reported in the 8p region defined by markers D8S133 to NEFL is greater than 80% in prostate cancer microdissected samples (Vocke et al., Cancer Res. 56:2411-2416, 1996). In addition, loss of 8p is also a frequent event in a number of other cancers including colon cancer, non-small cell lung cancer, breast cancer (Yaremko et al., Genes, Chrom. Cancer 16:189-195, 1996), head and neck cancer (Scholnick et al., J. Natl. Cancer Inst. 88:1676-1682, 1996), hepatocarcinoma (Emi et al., Genes, Chrom. Cancer 7:152-157, 1993), and bladder cancer (Takle et al., Oncogene 12:1083-1087, 1996). Linkage analyses on German breast cancer families' pedigrees have identified a strong linkage in this same region of 8p (Seitz et al., Oncogene 14:741-743, 1997), which has been termed the BRCA3 gene region (Kerangueven et al.).

Tango-63 has been mapped on the Stanford Human Genome Center G3 radiation hybrid panel close to marker D8S1734 with a LOD score of 6. The mapping was carried out using a pair of primers from the 3′ untranslated region (UTR). The primers are designated t63-f2 (5′-ATGTCATTGTTTTCACAGCA-3′; SEQ ID NO:12) and t63-r2 (5′-GCTCAAGCGATTCTCTCA-3′; SEQ ID NO:13). This map position is located in the most frequently lost region of chromosome 8 between markers D8S133 and NEFL.

Subsequently, three overlapping yeast artificial chromosomes (YACs) were used to place Tango-63 on the physical map of chromosome 8 between markers WI-6088 and WI-6563.

Deposit Information

Two plasmids bearing cDNA encoding Tango-63d and Tango-63e respectively, were deposited with the American Type Culture Collection (12301 Parklawn Drive, Rockville, Md. 20852-1776) on Feb. 13, 1997. The plasmid encoding Tango-63d was assigned accession number 98368, and the plasmid encoding Tango-63e was assigned accession number 98367.

The subject cultures have been deposited under conditions that assure that access to the cultures will be available during the pendency of the patent application to one determined by the Commissioner of Patents and Trademarks to be entitled thereto under 37 CFR 1.14 and 35 USC 122. The deposits are available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which can issue disclosing the cultures plus five years after the last request for a sample from the deposit. The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them.

Additional embodiments are within the following claims.

What is claimed is:

1. An isolated nucleic acid molecule comprising a nucleotide sequence encoding a polypeptide that is at least 85% identical to SEQ ID NO:2.

2. An isolated nucleic acid molecule comprising a nucleotide sequence encoding a polypeptide that is at least 85% identical to SEQ ID NO:4.

3. The nucleic acid molecule of claim 1 or claim 2, said molecule encoding a polypeptide that associates with the cell surface and mediates the cellular response to an apoptotic signal.

4. The nucleic acid molecule of claim 1, said molecule encoding the amino acid sequence of SEQ ID NO:2.

5. The nucleic acid molecule of claim 4, said molecule comprising the nucleotide sequence of SEQ ID NO:1.

6. The nucleic acid molecule of claim 2, said molecule encoding the amino acid sequence of SEQ ID NO:4.

7. The nucleic acid molecule of claim 6, said molecule comprising the nucleotide sequence of SEQ ID NO:3.

8. An isolated nucleic acid molecule, said molecule comprising the cDNA sequence contained within ATCC Accession No. 98367.

9. An isolated nucleic acid molecule, said molecule comprising the cDNA sequence contained within ATCC Accession No. 98368.

10. A vector comprising the nucleic acid molecule of claim 1, claim 2, claim 4, or claim 6.

11. The vector of claim 10, said vector being an expression vector.

12. The vector of claim 11, further comprising a regulatory element.

13. The vector of claim 12, wherein the regulatory element is selected from the group consisting of the cytomegalovirus hCMV immediate early gene, the early promoter of SV40 adenovirus, the late promoter of SV40 adenovirus, the lac system, the trp system, the TAC system, the TRC system, the major operator and promoter regions of phage λ, the control regions of fd coat protein, the promoter for 3-phosphoglycerate kinase, the promoters of acid phosphatase, and the promoters of the yeast α-mating factors.

14. The vector of claim 12, wherein said regulatory element directs tissue-specific expression.

15. The vector of claim 10, further comprising a reporter gene.

16. The vector of claim 15, wherein the reporter gene is selected from the group consisting of β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT).

17. The vector of claim 10, wherein said vector is a plasmid.

18. The vector of claim 10, wherein said vector is a virus.

19. The vector of claim 18, wherein said virus is a retrovirus.

20. A genetically engineered host cell comprising the expression vector of claim 11.

21. The cell of claim 20, wherein said cell is eukaryotic.

22. A substantially pure polypeptide having the amino acid sequence encoded by the nucleic acid molecule of claim 1, claim 2, claim 4, or claim 6.

23. The polypeptide of claim 22, further comprising a heterologous polypeptide other than a Caspase-8 polypeptide.

24. An antibody that specifically binds Tango-63d or Tango-63e.

25. The antibody of claim 24, wherein said antibody is a neutralizing antibody.

26. A transgenic animal harboring the nucleic acid molecule of claim 1, claim 2, claim 4, or claim 6.

27. A method of determining whether a patient has a disorder associated with an abnormal rate of apoptotic cell death, said method comprising quantitating the level of Tango-63d expression in a biological sample obtained from said patient.

28. The method of claim 27, comprising quantitating mRNA encoding Tango-63d.

29. The method of claim 27, comprising quantitating Tango-63d protein.

30. A method of determining whether a patient has a disorder associated with an abnormal rate of apoptotic cell death, said method comprising quantitating the level of Tango-63e expression in a biological sample obtained from said patient.

31. The method of claim 30, comprising quantitating mRNA encoding Tango-63e.

32. The method of claim 30, comprising quantitating Tango-63e protein.

33. The method of claim 28 or claim 31, comprising an RNAse protection assay, Northern blot analysis, or amplification by RT-PCR.

34. The method of claim 29 or claim 32, comprising Western blot analysis.

35. The method of claim 27 or claim 30, wherein said biological sample is a tumor sample.

36. A method of treating a patient who has a disorder associated with abnormal expression or activity of Tango-63d, said method comprising administering to the patient a compound that modulates the expression or activity of Tango-63d.

37. The method of claim 36, wherein the compound comprises a small molecule, an antisense nucleic acid molecule, or a ribozyme.

38. A method of treating a patient who has a disorder associated with abnormal expression or activity of Tango-63e, said method comprising administering to the patient a compound that modulates the expression or activity of Tango-63e.

39. The method of claim 38, wherein the compound comprises a small molecule, an antisense nucleic acid molecule, or a ribozyme.

40. A therapeutic composition comprising the compound of claim 36 or claim 38.

41. A method for treating a patient who has a disorder associated with abnormal activity of the Tango-63d receptor complex, said method comprising administering a compound that mediates oligomerization between Tango-63d and one or more of the polypeptides that form a Tango-63d receptor complex.

42. A method for treating a patient who has a disorder associated with abnormal activity of the Tango-63 e receptor complex, said method comprising administering a compound that modulates activity of said complex.

43. The method of claim 42, wherein said compound mediates oligomerization between Tango-63e and one or more of the polypeptides that form a Tango-63e receptor complex.

44. A method for treating a patient who has a disorder associated with abnormal expression of Tango-63e or a member of the Tango-63e receptor complex, said method comprising administering a compound that modulates expression of Tango-63e or a member of the Tango-63e complex.

45. The method of claim 41 or claim 42, wherein the patient has a disorder in which the rate of apoptotic cell death is abnormally low.

46. The method of claim 41 or claim 42, wherein the compound is synthetic.

47. A method of treating a patient who has a disorder associated with excessive apoptotic cell death, said method comprising administering to the patient the nucleic acid molecule of claim 1 or claim 2, wherein said molecule encodes a dysfunctional polypeptide.

48. A method of treating a patient who has a disorder associated with excessive apoptotic cell death, said method comprising administering to the patient the polypeptide of claim 22, wherein said polypeptide is dysfunctional.

49. A method of identifying a compound that modulates expression of Tango-63d, said method comprising assessing the expression of Tango-63d in the presence and absence of said compound.

50. A method of identifying a compound that modulates expression of Tango-63e, said method comprising assessing the expression of Tango-63e in the presence and absence of said compound.

51. A method for treating a patient who has a disease characterized by an abnormally low rate of apoptotic cell death, said method comprising administering a compound that mediates oligomerization between Tango-63d and one or more of the polypeptides that form a Tango-63d receptor complex.

52. A method for treating a patient who has a disease characterized by an abnormally low rate of apoptotic cell death, said method comprising administering a compound that mediates oligomerization between Tango-63e and one or more of the polypeptides that form a Tango-63e receptor complex.

53. A method of identifying a compound that modulates the activity of Tango-63d, said method comprising assessing the activity of Tango-63d in the presence and absence of said compound.

54. A method of identifying a compound that modulates the activity of Tango-63e, said method comprising assessing the activity of Tango-63e in the presence and absence of said compound.

55. A method for determining whether a selected compound modulates oligomerization between Tango-63d and one or more of the polypeptides that form a Tango-63d receptor complex, said method comprising measuring oligomerization of the Fas/APO-1 receptor complex and Tango-63d and one or more of the polypeptides that form a Tango-63d receptor complex in the presence and absence of said selected compound.

56. A method for determining whether a selected compound modulates oligomerization between Tango-63d and one or more of the polypeptides that form a Tango-63e receptor complex, said method comprising measuring oligomerization of Tango-63e and one or more of the polypeptides that from a Tango-63e receptor complex in the presence and absence of said selected compound.

57. An isolated nucleic acid molecule which hybridizes under stringent conditions to a nucleic acid molecule having the nucleotide sequence of SEQ ID NO:1, said isolated nucleic acid molecule encoding Tango-63d.

58. An isolated nucleic acid molecule which hybridizes under stringent conditions to a nucleic acid molecule having the nucleotide sequence of SEQ ID NO:3, said isolated nucleic acid molecule encoding Tango-63e.

59. An isolated nucleic acid molecule comprising a nucleotide sequence which is at least 90% identical to the nucleotide sequence of SEQ ID NO:1, said isolated nucleic acid molecule encoding Tango-63d.

60. An isolated nucleic acid molecule comprising a nucleotide sequence which is at least 90% identical to the nucleotide sequence of SEQ ID NO:3, said isolated nucleic acid molecule encoding Tango-63e.

61. The method of claim 47, wherein said dysfunctional polypeptide comprises a mutation that inhibits ligand binding.

62. The method of claim 47, wherein said dysfunctional polypeptide comprises a mutation that inhibits formation of a receptor complex.

63. A method of identifying a ligand capable of binding a polypeptide having an amino acid sequence encoded by the nucleic acid molecule of claim 1, claim 2, claim 4, or claim 6, said method comprising contacting said polypeptide with said ligand, and determining whether a complex forms between said ligand and said polypeptide.

64. An isolated nucleic acid molecule that hybridizes under stringent conditions to cDNA sequence contained within ATCC Accession No. 98367.

65. An isolated nucleic acid molecule that hybridizes under stringent conditions to cDNA sequence contained within ATCC Accession No. 98368.

66. An isolated nucleic acid molecule that is 85% identical to SEQ ID NO:1 (FIG. 1).

67. An isolated nucleic acid molecule that is 85% identical to SEQ ID NO:3 (FIG. 2).

68. An isolated nucleic acid molecule that is 95% identical to SEQ ID NO:1.

69. An isolated nucleic acid molecule that is 95% identical to SEQ ID NO:3.

70. An isolated nucleic acid molecule that is 85% identical to cDNA sequence contained within ATCC Accession No. 98367.

71. An isolated nucleic acid molecule that is 85% identical to cDNA sequence contained within ATCC Accession No. 98368.

72. An isolated nucleic acid molecule that is 95% identical to cDNA sequence contained within ATCC Accession No. 98367.

73. An isolated nucleic acid molecule that is 95% identical to cDNA sequence contained within ATCC Accession No. 98368.

74. An isolated nucleic acid molecule that hybridizes under stringent conditions to nucleotides 128 to 1447 of SEQ ID NO:1 (FIG. 1).

75. An isolated nucleic acid molecule that hybridizes under stringent conditions to nucleotides 128 to 1360 of SEQ ID NO:3 (FIG. 2).

76. The polypeptide encoded by the nucleic acid molecule of claim 64.

77. The polypeptide encoded by the nucleic acid molecule of claim 65.

78. The polypeptide encoded by the nucleic acid molecule of claim 66.

79. The polypeptide encoded by the nucleic acid molecule of claim 67.

80. The polypeptide encoded by the nucleic acid molecule of claim 68.

81. The polypeptide encoded by the nucleic acid molecule of claim 69.
